(19) United States
(12) Patent Application Publication
(10) Pub. No.: US 2026/0158143 A1
(43) Pub. Date: Jun. 11, 2026

(54) ANTI-MESOTHELIN ANTIBODY REAGENTS

(71) Applicant: The General Hospital Corporation, Boston, MA (US)

(72) Inventors: Marcela V. Maus, Lexington, MA (US); Stefanie Bailey, Boston, MA (US)

(73) Assignee: The General Hospital Corporation, Boston, MA (US)

(21) Appl. No.: 18/707,471

(22) PCT Filed: Nov. 4, 2022
(86) PCT No.: PCT/US2022/079282

§ 371 Date: May 3, 2024

Related U.S. Application Data
(60) Provisional application No. 63/275,866, filed on Nov. 4, 2021.

(51) Int. Cl. A61K 40/42; A61K 40/11; A61K 40/31; C07K 16/30; G01N 33/575

(52) U.S. Cl. CPC A61K 40/4255; A61K 40/11; A61K 40/31; C07K 16/30; G01N 33/5759

(57) ABSTRACT

The disclosure is directed to anti-mesothelin antibody reagents, nucleic acids encoding said reagents, cells comprising said antibody reagents and nucleic acids, and methods of treating cancer comprising administering the aforementioned to a subject.

### RELATED APPLICATION

This application claims the benefit under 35 U.S.C. § 119 (e) to U.S. Provisional Application No. 63/275,866, filed Nov. 4, 2021, entitled “Anti-Mesothelin Antibody Reagents” the entire contents of which are incorporated herein by reference.

### FEDERALLY SPONSORED RESEARCH

This invention was made with Government support under R01 CA 238268, awarded by the National Cancer Institute, and T32CA009216, awarded by the National Institute of Health. The Government has certain rights in the invention.

### REFERENCE TO AN ELECTRONIC SEQUENCE LISTING

The content of the electronic sequence listing (M105370027WO00-SEQ-ARM.xml; Size: 79,568 bytes; and Date of Creation: Nov. 3, 2022) is herein incorporated by reference in its entirety.

### BACKGROUND

The MSLN gene encodes a 71 kilodalton (kDa) precursor, which is a glycosylphosphatidylinositol-anchored membrane glycoprotein that is cleaved into two products: a soluble 31 kDa N-terminal protein called megakaryocyte potentiating factor (MPF) and a 40-kDa membrane-bound fragment called MSLN (mesothelin). Mesothelin is expressed on mesothelial cells and over-expressed on various cancers, including mesothelioma. Mesothelin can be used as a tumor antigen to detect cancer cells. Antibody reagents that bind mesothelin may be useful for diagnosing or treating cancers, and in the manufacture of therapeutics for treating cancers. There is a need for additional and alternative mesothelin targeted antibody reagents.

### SUMMARY

Described herein are antibody reagents and antibody conjugates that target mesothelin, which can be useful for treating a subject having a disease or disorder, e.g., a cancer, a plasma cell disease or disorder, or an autoimmune disease or disorder. Also provided herein are nucleic acids and vectors encoding said antibody reagents and conjugates, and cells comprising the antibody reagents, antibody conjugates, nucleic acids, or vectors described herein. Also provided herein are pharmaceutical compositions comprising antibody reagents and other compositions described herein. Further provided are methods of treating cancer in a subject by administering a composition described herein, and methods of detecting a mesothelin protein in a sample comprising contacting the sample with an antibody reagent or antibody conjugate described herein.

Accordingly, in one aspect, the disclosure is directed to an antibody reagent that binds to mesothelin or a portion thereof, comprising: (a) a heavy chain variable domain (VH) comprising three complementarity determining regions CDR-H1, CDR-H2, and CDR-H3, wherein the CDR-H1 comprises an amino acid sequence of SEQ ID NO: 13, or an amino acid sequence with no more than 1, 2, or 3 amino acid substitutions of SEQ ID NO: 13; the CDR-H2 comprises an amino acid sequence of SEQ ID NO: 14, or an amino acid sequence with no more than 1, 2, or 3 amino acid substitutions of SEQ ID NO: 14; and the CDR-H3 comprises an amino acid sequence of SEQ ID NO: 15, or an amino acid sequence with no more than 1, 2, or 3 amino acid substitutions of SEQ ID NO: 15, and (b) a light chain variable domain (VL) comprising three complementarity determining regions CDR-L1, CDR-L2, and CDR-L3, wherein the CDR-L1 comprises an amino acid sequence of SEQ ID NO: 16, or an amino acid sequence with no more than 1, 2, or 3 amino acid substitutions of SEQ ID NO: 16; the CDR-L2 comprises an amino acid sequence of SEQ ID NO: 17, or an amino acid sequence with no more than 1, 2, or 3 amino acid substitutions of SEQ ID NO: 17; and the CDR-L3 comprises an amino acid sequence of SEQ ID NO: 18, or an amino acid sequence with no more than 1, 2, or 3 amino acid substitutions of SEQ ID NO: 18.

In some embodiments, the antibody reagent comprises an antibody or an antibody fragment. In some embodiments, the antibody reagent is selected from the group consisting of an immunoglobulin molecule, a recombinant antibody, a disulfide linked Fv, a camelid antibody, a monoclonal antibody, a polyclonal antibody, a single-chain variable fragment (scFv), a chimeric antibody, a single domain antibody, a CDR-grafted antibody, a bispecific antibody, a diabody, an antigen binding fragment (Fab), a F(ab′)2, and a Fv. In some embodiments, the antibody is a human antibody or a humanized antibody. In some embodiments, the antibody reagent is an scFv.

In some embodiments, the antibody reagent comprises: (a) a heavy chain variable domain (VH) comprising three complementarity determining regions CDR-H1, CDR-H2, and CDR-H3, wherein the CDR-HI comprises an amino acid sequence of SEQ ID NO: 13; the CDR-H2 comprises an amino acid sequence of SEQ ID NO: 14; and the CDR-H3 comprises an amino acid sequence of SEQ ID NO: 15; and (b) a light chain variable domain (VL) comprising three complementarity determining regions CDR-L1, CDR-L2, and CDR-L3, wherein the CDR-L1 comprises an amino acid sequence of SEQ ID NO: 16; the CDR-L2 comprises an amino acid sequence of SEQ ID NO: 17; and the CDR-L3 comprises an amino acid sequence of SEQ ID NO: 18. In some embodiments, the antibody reagent comprises: (a) a heavy chain variable domain (VH) comprising three complementarity determining regions CDR-H1, CDR-H2, and CDR-H3, wherein the CDR-HI consists of an amino acid sequence of SEQ ID NO: 13; the CDR-H2 consists of an amino acid sequence of SEQ ID NO: 14; and the CDR-H3 consists of an amino acid sequence of SEQ ID NO: 15; and (b) a light chain variable domain (VL) comprising three complementarity determining regions CDR-L1, CDR-L2, and CDR-L3, wherein the CDR-L1 consists of an amino acid sequence of SEQ ID NO: 16; the CDR-L2 consists of an amino acid sequence of SEQ ID NO: 17; and the CDR-L3 consists of an amino acid sequence of SEQ ID NO: 18.

In some embodiments, the antibody reagent comprises a VH domain of the amino acid sequence of SEQ ID NO: 19 or an amino acid sequence having at least 90% sequence identity to the amino acid sequence of SEQ ID NO: 19. In some embodiments, the antibody reagent comprises a VL domain of the amino acid sequence of SEQ ID NO: 20, or an amino acid sequence having at least 90% sequence identity to the amino acid sequence of SEQ ID NO: 20. In some embodiments, the antibody reagent comprises a VH-VL domain of the amino acid sequence of SEQ ID NO: 21, or an amino acid sequence having at least 90% sequence identity to the amino acid sequence of SEQ ID NO: 21. In some embodiments, the antibody reagent comprises a VL-VH domain of the amino acid sequence of SEQ ID NO: 22, or an amino acid sequence having at least 90% sequence identity to the amino acid sequence of SEQ ID NO: 22.

In some embodiments, the mesothelin protein comprises an amino acid sequence of SEQ ID NO: 23.

In another aspect, the disclosure is directed to a bispecific antibody reagent comprising an antibody reagent described herein and a second antibody reagent. In some embodiments, the second antibody reagent binds to a tumor antigen. In some embodiments, the tumor antigen to which the second antibody reagent binds is a solid tumor antigen. In some embodiments, the second antibody reagent binds to a protein selected from the group consisting of: CD3, epidermal growth factor (EGFR), and a portion of either thereof. In some embodiments, a bispecific antibody comprises a cleavable moiety between an antibody reagent described herein and the second antibody reagent.

In another aspect, the disclosure is directed to an antibody conjugate comprising an antibody reagent described herein or a bi-specific antibody reagent described herein, and a conjugate moiety. In some embodiments, the conjugate moiety is a cytotoxic agent. In some embodiments, the cytotoxic agent is selected from the group consisting of ozogamicin, vedotin, bleomycin, an auristatin, a maytansinoid, calicheamicin, emtansine, deruxtecan, govitecan, mafodotin, duocarmazine, soravtansine, and tesirine. In some embodiments, the conjugate is a detectable moiety. In some embodiments, the detectable moiety comprises a fluorescent molecule, a chromogenic molecule, an affinity purification tag, a radioisotope, a heavy isotope, a nucleotide probe, or a nanoparticle.

In another aspect, the disclosure is directed to a nucleic acid molecule comprising a nucleic acid sequence encoding any one of the antibody reagents described herein or any one of the bispecific antibody reagents described herein. In some embodiments, the nucleic acid molecule is a vector or a plasmid.

In another aspect, the disclosure is directed to a viral vector comprising a nucleic acid molecule described herein. In some embodiments, the viral vector is an adenovirus associated vector, a lentiviral vector, or an oncolytic herpes simplex virus.

In another aspect, the disclosure is directed to a cell comprising an antibody reagent, a bispecific antibody reagent, an antibody conjugate, or a nucleic acid molecule described herein. In some embodiments, the cell is a human cell. In some embodiments, the human cell is an immune cell. In some embodiments, the cell is a cancer cell.

In another aspect, the disclosure is directed to a composition comprising an antibody reagent, a bispecific antibody reagent, an antibody conjugate, a nucleic acid molecule, or a cell described herein. In some embodiments, the composition comprises a therapeutically effective amount of the antibody reagent, the bispecific antibody reagent, the antibody conjugate, the nucleic acid molecule, the viral vector, or the cell. In some embodiments, the composition further comprising a pharmaceutically acceptable excipient.

In another aspect, the disclosure is directed to a method of treating cancer in a subject comprising administering to the subject an antibody reagent, a bispecific antibody reagent, an antibody conjugate, a nucleic acid molecule, a viral vector, a cell, or a composition described herein. In some embodiments, the cancer is characterized by the presence of one or more solid tumors. In some embodiments, the cancer expresses mesothelin. In some embodiments, the cancer is selected from the group consisting of mesothelioma, pancreatic cancer, ovarian cancer, lung cancer, endometrial cancer, biliary cancer, breast cancer, pleural cancer, colorectal cancer, appendiceal cancer, esophageal cancer, thymic cancer, cervical cancer, and gastric cancer. In some embodiments, the subject is a human.

In another aspect, the disclosure is directed to a method of detecting a mesothelin protein in a sample, comprising contacting the sample with an antibody conjugate described herein, e.g., comprising a detectable moiety. In some embodiments, the method comprises contacting the antibody conjugate with a sample. In some embodiments, the method of detecting the mesothelin protein comprises performing a Western blot, immunohistochemistry, immunocytochemistry, Enzyme Linked Immunosorbent Assay, fluorescent microscopy, flow cytometry, or Fluorescence activated cell sorting (FACS) colorimetry. In some embodiments, the sample comprises mesothelin. In some embodiments, the method further comprises administering the antibody conjugate to a subject. In some embodiments, detecting the antibody conjugate in a subject comprises using magnetic resonance imagining, ultrasound, computerized tomography scan, positron emission tomography, or single-photon emission computerized tomography. In some embodiments, the method further comprises collecting a sample from the subject.

In another aspect, the disclosure is directed to an antibody reagent that binds to mesothelin, wherein the antibody reagent binds to an epitope in region III of mesothelin. In some embodiments, the antibody reagent binds to an epitope comprising the amino acid sequence of RLAFQNMNGSEY (SEQ ID NO: 37).

For convenience, the meaning of some terms and phrases used in the specification, examples, and appended claims, are provided below. Unless stated otherwise, or implicit from context, the following terms and phrases include the meanings provided below. The meanings are provided to aid in describing particular embodiments, and are not intended to limit the claimed technology, because the scope of the technology is limited only by the claims. Unless otherwise defined, all technical and scientific terms used herein have the same meaning as commonly understood by one of ordinary skill in the art to which this technology belongs. If there is an apparent discrepancy between the usage of a term in the art and its meaning provided herein, the meaning provided within the specification shall prevail.

As used herein, the term “antibody reagent” refers to a polypeptide that includes at least one immunoglobulin variable domain or immunoglobulin variable domain sequence and which specifically binds a given antigen. An antibody reagent can comprise an antibody (e.g., a full length antibody) or a polypeptide comprising an antigen-binding domain of an antibody. In some embodiments of any of the aspects, an antibody reagent can comprise a monoclonal antibody or a polypeptide comprising an antigen-binding domain of a monoclonal antibody. For example, an antibody reagent can include a heavy (H) chain variable region (abbreviated herein as VH), and a light (L) chain variable region (abbreviated herein as VL). In another example, an antibody reagent includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, CDRs, and domain antibody (dAb) fragments (see, e.g., de Wildt et al., Eur. J. Immunol. 26(3): 629-639, 1996; which is incorporated by reference herein in its entirety)) as well as complete antibodies. The term “antibody reagent” encompasses multispecific antibodies, e.g., bispecific antibodies. An antibody reagent can have the structural features of IgA, IgG, IgE, IgD, or IgM (as well as subtypes and combinations thereof). An antibody reagent may comprise antibodies from any source, including mouse, rabbit, pig, rat, and primate (human and non-human primate) and primatized antibodies. An antibody reagent may also include midibodies, humanized antibodies, chimeric antibodies, and the like. Fully human antibody binding domains can be selected, for example, from phage display libraries using methods known to those of ordinary skill in the art. Furthermore, antibody reagents include single domain antibodies, such as camelid antibodies. In some embodiments, an antibody reagent comprises a nanobody derived from shark antibody. In some embodiments, an antibody reagent comprises a diabody. In some embodiments, an antibody reagent comprises a framework having a human germline sequence. In another embodiment, an antibody reagent comprises a heavy chain constant domain selected from the group consisting of IgG, IgG1, IgG2, IgG2A, IgG2B, IgG2C, IgG3, IgG4, IgA1, IgA2, IgD, IgM, and IgE constant domains. In some embodiments, an antibody reagent comprises a constant domain, e.g., an Fc region. An immunoglobulin constant domain refers to a heavy or light chain constant domain. Human IgG heavy chain and light chain constant domain amino acid sequences and their functional variations are known. With respect to the heavy chain, in some embodiments, the heavy chain of an antibody reagent described herein can be an alpha (α), delta (δ), epsilon (ε), gamma (γ) or mu (μ) heavy chain. In some embodiments, the heavy chain of an antibody described herein can comprise a human alpha (α), delta (δ), epsilon (ε), gamma (γ) or mu (μ) heavy chain. In some embodiments, an antibody reagent described herein comprises a human gamma 1 CH1, CH2, and/or CH3 domain. In some embodiments, the amino acid sequence of the VH domain comprises the amino acid sequence of a human gamma (γ) heavy chain constant region, such as any known in the art. Non-limiting examples of human constant region sequences have been described in the art, e.g., see U.S. Pat. No. 5,693,780 and Kabat E A et al., (1991) supra. In some embodiments, an antibody reagent comprises a polypeptide comprising one or more antigen binding fragments of the disclosure linked to a linker polypeptide or an immunoglobulin constant domain. Linker polypeptides (e.g., a linker described herein) may comprise two or more amino acid residues joined by peptide bonds and are used to link one or more antigen binding portions. An antibody reagent may be part of a larger immunoadhesion molecule, formed by covalent or noncovalent association of an antibody or portion thereof with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058).

The terms “anti-mesothelin antibody,” “an antibody that binds to mesothelin,” and “an antibody that specifically binds to mesothelin” refer to an antibody that is capable of binding mesothelin with sufficient affinity such that the antibody is useful as a preventative, diagnostic, and/or therapeutic agent in targeting mesothelin. In one embodiment, the extent of binding of an anti-mesothelin antibody to an unrelated, non-mesothelin protein is less than about 10% of the binding of the antibody to mesothelin as measured, e.g., by a radioimmunoassay (RIA). In certain embodiments, an antibody that binds to mesothelin has a dissociation constant (K_{D}) of ≤1 μM, ≤100 nM, ≤10 nM, ≤1 nM, or ≤0.1 nM, (e.g. 10⁻⁶, 10⁻⁷, or 10⁻⁸ M or less, e.g. from 10⁻⁶ M to 10⁻⁹ M). In certain embodiments, an antibody that binds to mesothelin has a dissociation constant (K_{D}) of ≤1 nM, ≤0.1 nM, ≤0.01 nM, or ≤0.001 nM, (e.g. 10⁻⁹, 10⁻¹⁰, 10⁻¹¹, or 10⁻¹² M or less, e.g. from 10⁻⁹ M to 10⁻¹² M).

As used herein, the term “CDR” refers to the complementarity determining region within antibody variable sequences. There are three CDRs in each of the variable regions of the heavy chain and the light chain, which are designated CDR1, CDR2 and CDR3, for each of the variable regions. The term “CDR set” as used herein refers to a group of three CDRs that occur in a single variable region capable of binding the antigen. The exact boundaries of these CDRs have been defined differently according to different systems. The system described by Kabat (Kabat et al., Sequences of Proteins of Immunological Interest (National Institutes of Health, Bethesda, Md. (1987) and (1991)) not only provides an unambiguous residue numbering system applicable to any variable region of an antibody, but also provides precise residue boundaries defining the three CDRs. These CDRs may be referred to as Kabat CDRs. Sub-portions of CDRs may be designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan (FASEB J. 9:133-139 (1995)) and MacCallum (J Mol Biol 262 (5): 732-45 (1996)). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia defined CDRs.

As used herein, the term “CDR-grafted antibody” refers to antibodies which comprise heavy and light chain variable region sequences from one species but in which the sequences of one or more of the CDR regions of VH and/or VL are replaced with CDR sequences of another species, such as antibodies having murine heavy and light chain variable regions in which one or more of the murine CDRs (e.g., CDR3) has been replaced with human CDR sequences.

As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, CDR-L2, and CDR-L3 of light chain and CDR-H1, CDR-H2, and CDR-H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FRI, FR2, FR3 or FR4, a framework region, as referred by others, represents the combined FRs within the variable region of a single, naturally occurring immunoglobulin chain. As used herein, a FR represents one of the four sub-regions, and FRs represents two or more of the four sub-regions constituting a framework region. Human heavy chain and light chain acceptor sequences are known in the art. In one embodiment, the acceptor sequences known in the art may be used in the antibodies disclosed herein.

As used herein, the term “human antibody” refers to antibodies having variable and constant regions derived from human germline immunoglobulin sequences. The human antibodies of the disclosure may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include antibodies in which CDR sequences derived from the germline of another mammalian species, such as a mouse, have been grafted onto human framework sequences.

As used herein, the term “humanized antibody” refers to antibodies which comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. In one embodiment, humanized mesothelin antibody and antigen binding portions are provided. Such antibodies may be generated by obtaining murine mesothelin monoclonal antibodies using traditional hybridoma technology followed by humanization using in vitro genetic engineering, such as those disclosed in Kasaian et al. PCT publication No. WO 2005/123126 A2.

An “isolated antibody”, as used herein, is intended to refer to an antibody that is substantially free of other antibodies having different antigenic specificities (e.g., an isolated antibody that specifically binds mesothelin is substantially free of antibodies that specifically bind antigens other than mesothelin). An isolated antibody that specifically binds mesothelin may, however, have cross-reactivity to other antigens, such as mesothelin from other species. Moreover, an isolated antibody may be substantially free of other cellular material and/or chemicals.

As used herein, the term “specifically binds” refers to the ability of a molecule to bind to a binding partner with a degree of affinity or avidity that enables the molecule to be used to distinguish the binding partner from an appropriate control in a binding assay or other binding context. With respect to an antibody, the term, “specifically binds”, refers to the ability of the antibody to bind to a specific antigen with a degree of affinity or avidity, compared with an appropriate reference antigen or antigens, that enables the antibody to be used to distinguish the specific antigen from others, e.g., to an extent that permits preferential targeting to certain cells, e.g., muscle cells, through binding to the antigen, as described herein. In some embodiments, an antibody specifically binds to a target if the antibody has a K_{D} for binding the target of at least about 10⁻⁴ M, 10⁻⁵ M, 10⁻⁶ M, 10⁻⁷ M, 10⁻⁸ M, 10⁻⁹ M, 10⁻¹⁰ M, 10⁻¹¹ M, 10⁻¹² M, 10⁻¹³ M, or less.

As used herein, a “subject” means a human or animal. Usually the animal is a vertebrate such as a primate, rodent, domestic animal or game animal. Primates include, for example, chimpanzees, cynomolgus monkeys, spider monkeys, and macaques, e.g., rhesus. Rodents include, for example, mice, rats, woodchucks, ferrets, rabbits and hamsters. Domestic and game animals include, for example, cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient,” and “subject” are used interchangeably herein. A subject can be one who has been previously diagnosed with or identified as suffering from or having a condition in need of treatment (e.g., mesothelioma, or another type of cancer, among others) or one or more complications related to such a condition, and optionally, have already undergone treatment for the condition or the one or more complications related to the condition. Alternatively, a subject can also be one who has not been previously diagnosed as having such condition or related complications. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “tumor antigen” and “cancer antigen” are used interchangeably to refer to antigens that are differentially expressed by cancer cells and can thereby be exploited in order to target cancer cells. Cancer antigens are antigens that can potentially stimulate apparently tumor-specific immune responses or may be used to selectively target a molecule (e.g., an antibody reagent or antibody conjugate) to the tumor. Some of these antigens are encoded, although not necessarily expressed, by normal cells. These antigens can be characterized as those which are normally silent/not expressed in normal cells, those that are expressed only at certain stages of differentiation and those that are temporally expressed such as embryonic and fetal antigens. Other cancer antigens are encoded by mutant cellular genes, such as oncogenes (e.g., activated Ras oncogene), suppressor genes (e.g., mutant p53), and fusion proteins resulting from internal deletions or chromosomal translocations. Still other cancer antigens can be encoded by viral genes such as those carried on RNA and DNA tumor viruses. Many tumor antigens have been defined in terms of multiple solid tumors: MAGE 1, 2, & 3, defined by immunity; MART-1/Melan-A, gp100, carcinoembryonic antigen (CEA), human epidermal growth factor receptor (HER2), mucins (e.g., MUC-1), prostate-specific antigen (PSA), and prostatic acid phosphatase (PAP). In addition, viral proteins such as some encoded by hepatitis B (HBV), Epstein-Barr (EBV), and human papilloma (HPV) have been shown to be important in the development of hepatocellular carcinoma, lymphoma, and cervical cancer, respectively. Examples of tumor antigens are provided below and include, e.g., mesothelin, EGFR, EGFRvIII, CD19, PSMA, B cell maturation antigen (BCMA), interleukin-13 receptor subunit alpha-2 (IL 13Ra2), etc. In some embodiments, a tumor antigen is a tumor-associated antigen. A tumor-associated antigen can be present proximal to cancer cells or a tumor, but not necessarily attached to the cancer cell or a tumor. For example, a tumor-associated antigen may be a marker present (e.g., at an increased level relative to a normal tissue) in the extracellular matrix surrounding or adjacent to a tumor.

As used herein, “Treg antigen” or “Treg-associated antigen” is used interchangeably to refer to antigens that are expressed by T regulatory (Treg) cells. These antigens may optionally be targeted by the antibody reagents, antibody conjugates, and methods of the disclosure. Examples of Treg antigens are provided below and include, e.g., GARP, LAP, CD25, and CTLA-4. In some embodiments, the antibody reagent targeting GARP comprises SEQ ID NO: 11. In some embodiments, the antibody reagent targeting GARP is a camelid antibody or single domain antibody. In some embodiments, the antibody reagent targeting LAP comprises SEQ ID NO: 12. In some embodiments, the antibody reagent targeting LAP comprises the VH and VL domain of SEQ ID NO: 12. In some embodiments, the antibody reagent targeting LAP comprises from N-terminal to C-terminal a VH domain then a VL domain. In some embodiments, the antibody reagent targeting LAP comprises from N-terminal to C-terminal a VL domain then a VH domain.

As used herein, the term “chimeric” refers to the product of the fusion of portions of at least two or more different polynucleotide molecules. In one embodiment, the term “chimeric” refers to a gene expression element produced through the manipulation of known elements or other polynucleotide molecules.

As used herein, the term “T cell engaging antibody molecules” or “TEAMs” is meant polypeptides that each include tandemly linked single-chain variable fragments (scFvs). Optionally, the scFvs are linked by a linker (e.g., a glycine-rich linker). One scFv of the TEAM binds to a T cell surface antigen (e.g., the T cell receptor (TCR) or a portion thereof (e.g., to the CD3ε subunit)) and the other binds to a target antigen (e.g., a tumor antigen). In some embodiments, the T cell surface antigen is a Treg antigen.

In some embodiments, “activation” can refer to the state of a T cell that has been sufficiently stimulated to induce detectable cellular proliferation. In some embodiments, activation can refer to induced cytokine production. In other embodiments, activation can refer to detectable effector functions. At a minimum, an “activated T cell” as used herein is a proliferative T cell.

As used herein, the term “therapeutically effective amount” refers to the amount necessary or sufficient to realize a desired biologic effect. For example, a therapeutically effective amount of a CAR, CAR T cell, or antibody reagent of the present disclosure may be that amount sufficient to ameliorate one or more symptoms of a cancer. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject. The effective amount for any particular application can vary depending on such factors as the disease or condition being treated, the particular therapeutic compounds being administered the size of the subject, or the severity of the disease or condition. One of ordinary skill in the art can empirically determine the effective amount of a particular therapeutic compound associated with the present disclosure without necessitating undue experimentation.

As used herein, the terms “specific binding” and “specifically binds” refer to a physical interaction between two molecules, compounds, cells and/or particles wherein the first entity binds to the second, target, entity with greater specificity and affinity than it binds to a third entity which is a non-target. In some embodiments, specific binding can refer to an affinity of the first entity for the second target, entity, which is at least 10 times, at least 50 times, at least 100 times, at least 500 times, at least 1000 times or more greater than the affinity for the third non-target entity under the same conditions. A reagent specific for a given target is one that exhibits specific binding for that target under the conditions of the assay being utilized. A non-limiting example includes an antibody, or a ligand, which recognizes and binds with a cognate binding partner (for example, a stimulatory and/or costimulatory molecule present on a T cell) protein.

As used herein, the term “operably linked” refers to a first polynucleotide molecule, such as a promoter, connected with a second transcribable polynucleotide molecule, such as a gene of interest, where the polynucleotide molecules are so arranged that the first polynucleotide molecule affects the function of the second polynucleotide molecule. The two polynucleotide molecules may or may not be part of a single contiguous polynucleotide molecule and may or may not be adjacent. For example, a promoter is operably linked to a gene of interest if the promoter regulates or mediates transcription of the gene of interest in a cell.

In some embodiments, a polypeptide described herein (or a nucleic acid encoding such a polypeptide) can be a functional fragment of one of the amino acid sequences described herein. As used herein, a “functional fragment” is a fragment or segment of a peptide that retains at least 50% of the wildtype reference polypeptide's activity according to an assay known in the art or described below herein. A functional fragment can comprise conservative substitutions of the sequences disclosed herein.

As used herein, the term “variant” refers to a polypeptide substantially homologous to a native or reference polypeptide, but which has an amino acid sequence different from that of the native or reference polypeptide because of one or a plurality of deletions, insertions, or substitutions. In some embodiments, a polypeptide described herein can be a variant of a polypeptide or molecule as described herein. In some embodiments, a polypeptide described herein is a variant of an antibody reagent described herein. In some embodiments, the variant is a conservatively modified variant. Conservative substitution variants can be obtained by mutations of native nucleotide sequences, for example. Variant polypeptide-encoding DNA sequences encompass sequences that comprise one or more additions, deletions, or substitutions of nucleotides when compared to a native or reference DNA sequence, but that encode a variant protein or fragment thereof that retains activity of the non-variant polypeptide. A wide variety of PCR-based site-specific mutagenesis approaches are known in the art and can be applied by the ordinarily skilled artisan. A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a variant sequence can be determined, for example, by comparing the two sequences using freely available computer programs commonly employed for this purpose on the world wide web (e.g., BLASTp or BLASTn with default settings).

As used herein, the term “polynucleotide” is used interchangeably with “nucleic acid” to indicate a polymer of nucleosides. Typically a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. However, the term encompasses molecules comprising nucleosides or nucleoside analogs containing chemically or biologically modified bases, modified backbones, etc., whether or not found in naturally occurring nucleic acids, and such molecules may be preferred for certain applications. Where this application refers to a polynucleotide it is understood that both DNA, RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically characterizes a specific nucleic acid. A polynucleotide sequence presented herein is presented in a 5′ to 3′ direction unless otherwise indicated.

As used herein, the term “polypeptide” refers to a polymer of amino acids. The terms “protein” and “polypeptide” are used interchangeably herein. A peptide is a relatively short polypeptide, typically between about 2 and 60 amino acids in length. Polypeptides used herein typically contain amino acids such as the 20 L-amino acids that are most commonly found in proteins. However, other amino acids and/or amino acid analogs known in the art can be used. One or more of the amino acids in a polypeptide may be modified, for example, by the addition of a chemical entity such as a carbohydrate group, a phosphate group, a fatty acid group, a linker for conjugation, functionalization, etc. A polypeptide that has a non-polypeptide moiety covalently or noncovalently associated therewith is still considered a “polypeptide.” Exemplary modifications include glycosylation and palmitoylation. Polypeptides can be purified from natural sources, produced using recombinant DNA technology or synthesized through chemical means such as conventional solid phase peptide synthesis, etc. The term “polypeptide sequence” or “amino acid sequence” as used herein can refer to the polypeptide material itself and/or to the sequence information (i.e., the succession of letters or three letter codes used as abbreviations for amino acid names) that biochemically characterizes a polypeptide. A polypeptide sequence presented herein is presented in an N-terminal to C-terminal direction unless otherwise indicated.

As used herein, the term “recombinant antibody”, as used herein, is intended to include all antibodies that are prepared, expressed, created or isolated by recombinant means, such as antibodies expressed using a recombinant expression vector transfected into a host cell (described in more details in this disclosure), antibodies isolated from a recombinant, combinatorial antibody library (Hoogenboom H. R., (1997) TIB Tech. 15:62-70; Azzazy H., and Highsmith W. E., (2002) Clin. Biochem. 35:425-445; Gavilondo J. V., and Larrick J. W. (2002) BioTechniques 29:128-145; Hoogenboom H., and Chames P. (2000) Immunology Today 21:371-378), antibodies isolated from an animal (e.g., a mouse) that is transgenic for the immunoglobulin genes (see e.g., Taylor, L. D., et al. (1992) Nucl. Acids Res. 20:6287-6295; Kellermann S-A., and Green L. L. (2002) Current Opinion in Biotechnology 13:593-597; Little M. et al (2000) Immunology Today 21:364-370) or antibodies prepared, expressed, created or isolated by any other means that involves splicing of immunoglobulin gene sequences to other DNA sequences. In some embodiments, the recombinant antibody is a recombinant human antibody. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. One embodiment of the disclosure provides fully human antibodies capable of binding human mesothelin which can be generated using techniques well known in the art, such as, but not limited to, using human Ig phage libraries such as those disclosed in Jermutus et al., PCT publication No. WO 2005/007699 A2.

As used herein, the term “vector,” refers to a nucleic acid construct capable of delivery to a host cell or transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, artificial chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example, in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′ UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring nucleic acids into cells either in vitro or in vivo. Numerous forms of viral vectors are known in the art.

By “recombinant vector” is meant a vector that includes a heterologous nucleic acid sequence or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra-chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down, or stop the progression or severity of a condition associated with a disease or disorder, e.g., mesothelioma, pancreatic cancer, ovarian cancer, lung cancer, endometrial cancer, biliary cancer, gastric cancer, breast cancer, pleural cancer, colorectal cancer, appendiceal cancer, esophageal cancer, thymic cancer, or cervical cancer, or other cancer, disease, or disorder. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized state of disease, delay or slowing of disease progression, amelioration or palliation of the disease state, remission (whether partial or total), and/or decreased mortality, whether detectable or undetectable. The term “treatment” of a disease also includes providing relief from the symptoms or side effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier in which the active ingredient would not be found to occur in nature.

As used herein, the terms “about” or “approximately” when used in connection with a quantity refer to a range of values around said quantity of ±1, 2, 3, 4, 5, 6, 7, 8, 9, or 10%, e.g., ±1% or ±10%.

As used herein, the singular terms “a,” “an,” and “the” include plural referents unless context clearly indicates otherwise. Similarly, the word “or” is intended to include “and” unless the context clearly indicates otherwise.

### BRIEF DESCRIPTION OF DRAWINGS

The accompanying drawings are not intended to be drawn to scale. The patent or application file contains at least one drawing executed in color. Copies of this patent or patent application publication with color drawing(s) will be provided by the Office upon request and payment of the necessary fee.

FIG. 1 shows a graph of ELISA data measuring binding of two exemplary anti-mesothelin antibody reagents of the disclosure to wells of a plate coated in mesothelin.

FIGS. 2A-2F show a western blot image, flow cytometry data, and fluorescence microscopy images characterizing binding of an exemplary anti-mesothelin antibody reagent of the disclosure, MGHmeso1, to various cell lines. FIG. 2A shows a western blot image showing an assay for binding of MGHmeso1 to lysates from cell lines Jeko-1, AsPC-1, BxPC-3, Capan-2, Panc-1, PDAC3, NCI-H226, OVCAR3, and OVCAR4. Molecular weight markers identify mesothelin precursor and mesothelin cleaved positions, with GAPDH as a membrane probe control. FIG. 2B shows graphs of flow cytometry data in an assay for binding of MGHmeso1 to Jeko-1, AsPC-1, BxPC-3, Capan-2, Panc-1, and NCI-H226 cells. FIG. 2C shows fluorescence micrographs showing an assay for binding of MGHmeso1 to AsPC-1, BxPC-3, Capan-2, or Panc-1 cells, with DAPI stained images at left, MGHmeso1 stained images in the middle, and merged (DAPI and MGHmeso1) images at right. FIG. 2D shows control fluorescence micrograph where AsPC-1 cells were given secondary antibody only. FIG. 2E shows fluorescence micrographs showing an assay for binding of MGHmeso1 to NCI-H226 cells, with DAPI stained images at left, MGHmeso1 stained images in the middle, and merged (DAPI and MGHmeso1) images at right. FIG. 2F shows control fluorescence micrograph where NCI-H226 cells were given secondary antibody only.

FIGS. 3A-3F show graphs of cytokine secretion and cell killing from CAR T cells comprising a CAR comprising an exemplary anti-mesothelin antibody reagent of the disclosure, MGHmeso1, or a CAR comprising a previously known anti-mesothelin antibody, SS1. Legend: NA, no transfection; PMA/ionomycin, positive control; Meso, mesothelin; and CD19. FIG. 3A shows levels of IFN-gamma, FIG. 3B shows levels of IL-2, FIG. 3C shows levels of GM-CSF, FIG. 3D shows levels of TNF-alpha, and FIG. 3E shows levels of Granzyme B. FIGS. 3E-3F show graphs of lysis of Capan-2 pancreatic cancer cells (mesothelin positive) (FIG. 3E) or JeKo-1 lymphoma cells (mesothelin negative) (FIG. 3F) upon exposure to untransfected cells (dotted line), CAR T cells comprising a CAR comprising an MGHmeso1 or SS1.

FIGS. 4A-4B show the epitope mapping of MGHmeso1. FIG. 4A shows a micro-array of binding of MGHmeso1 to mesothelin fragments. FIG. 4B shows a quantification of MGHmeso1 binding intensity to different epitopes of mesothelin in the microarray.

FIGS. 5A-5D shows that MGHmeso1 antibodies do not block binding of SSI comprising CARs to mesothelin epitopes indicating the SSI and MGHmeso1 bind different mesothelin epitopes. FIG. 5A shows that #CAR-T cells containing SSI or MGHmeso1 constructs have similar toxicity Capan-2 cell lines. FIG. 5B shows that #CAR-T cells containing SS1 or MGHmeso1 constructs have similar toxicity against Panc-1 cell lines. FIGS. 5A-5B were performed using an ACEA assay at a 1:1 ratio and cytolysis was measured over time. NT-no treatment, UTD=untransduced. FIGS. 5C-5D show CAR-T cells containing SSI or MGHmeso1 constructs were generated and tested against AsPC-1 cell using an ACEA assay at a 1:1 ratio. CAR-T and AsPC-1 cultures were given no treatment (NT) or treated with 3 ug/ml IgG or MGHmeso1 mAb and cytolysis was measured over time. FIG. 5C show that MGHmeso1 antibody treatment does not decrease the efficacy of SSI CAR T cells. FIG. 5D show that MGHmeso1 antibody treatment does decrease the efficacy of MGHmeso1 CAR T cell therapy.

### DETAILED DESCRIPTION

Provided herein are antibody reagents and antibody conjugates that bind to a mesothelin protein or a portion thereof. Without wishing to be bound by theory, MSLN (uniprot.org/uniprot/Q13421) is expressed on normal mesothelial cells in some tissues (e.g., pleura, pericardium, peritoneum) and in trace amounts in some epithelial cells (e.g., ovary, tunica vaginalis, rete testis, and fallopian tube), but is abundantly expressed in various cancer cells. See, e.g., Lv, Jiang, and Li, Peng. “Mesothelin as a biomarker for targeted therapy”. Biomark Res. 2019; 7:18; and Hassan et al. “Mesothelin Immunotherapy for Cancer: Ready for Prime Time?” J Clin Oncol. 2016 Dec. 1; 34 (34): 4171-4179, each of which is hereby incorporated by reference. Mesothelin may be used as a marker for cells associated with various cancers (e.g., over-expressed in various cancers), and antibody reagents and antibody conjugates that bind to mesothelin or a portion thereof may be used to target cells comprising mesothelin (e.g., to identify the presence of cells comprising mesothelin or to treat a mesothelin associated disease).

### Antibody Reagents

The disclosure provides, in part, antibody reagents that specifically bind to mesothelin or a portion thereof. Mesothelin sequences are known for a number of species, e.g., human mesothelin (NCBI Gene ID: 10232) and polypeptide (e.g., pre-protein NCBI Ref Seq: NP_005814.2). Mesothelin can refer to human mesothelin, including naturally occurring variants, molecules, and alleles thereof. Homologs and/or orthologs of human mesothelin are readily identified for such species by one of skill in the art, e.g., using the mesothelin ortholog search function or searching available sequence data for a given species for sequence similar to a reference mesothelin sequence. In some embodiments, an antibody reagent that specifically binds mesothelin specifically binds a homolog and/or ortholog of human mesothelin, e.g., in addition to human mesothelin.

In some embodiments, an antibody reagent is one, two, three, or all of monoclonal, human, humanized, or chimeric. An antibody reagent may comprise one or more full-length antibodies or antibody fragments thereof (e.g., an antibody fragment that binds mesothelin). The antibody fragment may be selected from the group consisting of Fab, Fab′-SH, Fv, scFv, and (Fab′) 2 fragments. In some instances, the antibody is an IgG antibody (e.g., an IgG1 antibody). Such antibodies may have a half-life of ≥3 days (e.g., ≥1 week, e.g., ≥2 weeks, e.g., ≥1 month, e.g., ≥2 months, e.g., ≥3 months, e.g., ≥4 months, e.g., ≥5 months, e.g., ≥6 months).

Amino acid sequences of an exemplary anti-mesothelin antibody reagent are provided below.

TABLE 1
Exemplary Anti-Mesothelin Antibody Reagent

| Sequence Name | Sequence and SEQ ID NO |
|---|---|
| CDR-H1 | NYRMH (SEQ ID NO: 13) |
| CDR-H2 | GIYPGNSDTNYNQKFKD (SEQ ID NO: 14) |
| CDR-H3 | GIRGSYFDY (SEQ ID NO: 15) |
| CDR-L1 | KASQSVDYDGDSYMN (SEQ ID NO: 16) |
| CDR-L2 | AASNLES (SEQ ID NO: 17) |
| CDR-L3 | QQSNEDPST (SEQ ID NO: 18) |
| VH | MECNWILPFILSVTSGVYSEILLQQTGTVLARPGTSVKMSCKASGYTFTNY |
| | RMHWVKQRPGQGLEWIGGIYPGNSDTNYNQKFKDKAKLTAVTSTSTANMEL |
| | SSLTNEDSAVYYCLRGIRGSYFDYWGQGTTLTVSS (SEQ ID NO: 19) |
| VL | METDTILLWVLLLWVPGSTGDIVLTQSPASLAVSLGQRATISCKASQSVDY |
| | DGDSYMNWYQQKPGQPPKLLIYAASNLESGIPARFSGSGSGTDFTLNIHPV |
| | EEEDAATYYCQQSNEDPSTFGGGTKLEVK (SEQ ID NO: 20) |
| VH-VL | MECNWILPFILSVTSGVYSEILLQQTGTVLARPGTSVKMSCKASGYTFTNY |
| | RMHWVKQRPGQGLEWIGGIYPGNSDTNYNQKFKDKAKLTAVTSTSTANMEL |
| | SSLTNEDSAVYYCLRGIRGSYFDYWGQGTTLTVSSGGGGSGGGGSGGGGSG |
| | GGGSMETDTILLWVLLLWVPGSTGDIVLTQSPASLAVSLGQRATISCKASQ |
| | SVDYDGDSYMNWYQQKPGQPPKLLIYAASNLESGIPARFSGSGSGTDFTLN |
| | IHPVEEEDAATYYCQQSNEDPSTFGGGTKLEVK (SEQ ID NO: 21) |
| VL-VH | METDTILLWVLLLWVPGSTGDIVLTQSPASLAVSLGQRATISCKASQSVDY |
| | DGDSYMNWYQQKPGQPPKLLIYAASNLESGIPARFSGSGSGTDFTLNIHPV |
| | EEEDAATYYCQQSNEDPSTFGGGTKLEVKGGGGSGGGGSGGGGSGGGGSME |
| | CNWILPFILSVTSGVYSEILLQQTGTVLARPGTSVKMSCKASGYTFTNYRM |
| | HWVKQRPGQGLEWIGGIYPGNSDTNYNQKFKDKAKLTAVTSTSTANMELSS |
| | LTNEDSAVYYCLRGIRGSYFDYWGQGTTLTVSS (SEQ ID NO: 22) |

In some embodiments, the antibody reagent comprises a heavy chain variable domain (VH) comprising three complementarity determining regions: CDR-H1, CDR-H2, and CDR-H3. In some embodiments, CDR-H1 comprises an amino acid sequence of SEQ ID NO: 13 or an amino acid sequence with no more than 1, 2, or 3 amino acid substitutions relative thereto. In some embodiments, CDR-H2 comprises an amino acid sequence of SEQ ID NO: 14 or an amino acid sequence with no more than 1, 2, or 3 amino acid substitutions relative thereto. In some embodiments, CDR-H3 comprises an amino acid sequence of SEQ ID NO: 15 or an amino acid sequence with no more than 1, 2, or 3 amino acid substitutions relative thereto. In some embodiments, the 1, 2, or 3 amino acid substitutions to CDR-H1, CDR-H2, and/or CDR-H3 are conservative amino acid substitutions.

In some embodiments, the antibody reagent comprises a heavy chain variable domain (VL) comprising three complementarity determining regions: CDR-L1, CDR-L2, and CDR-L3. In some embodiments, CDR-L1 comprises an amino acid sequence of SEQ ID NO: 16 or an amino acid sequence with no more than 1, 2, or 3 amino acid substitutions relative thereto. In some embodiments, CDR-L2 comprises an amino acid sequence of SEQ ID NO: 17 or an amino acid sequence with no more than 1, 2, or 3 amino acid substitutions relative thereto. In some embodiments, CDR-L3 comprises an amino acid sequence of SEQ ID NO: 18 or an amino acid sequence with no more than 1, 2, or 3 amino acid substitutions relative thereto. In some embodiments, the 1, 2, or 3 amino acid substitutions to CDR-L1, CDR-L2, and/or CDR-L3 are conservative amino acid substitutions.

In some embodiments, the antibody reagent comprises a heavy chain variable domain (VH) sequence having at least 80% sequence identity (e.g., at least 81%, 82%, 83%, 84%, 85%, 86%, 87%, 88%, 89%, 90%, 91%, 92%, 93%, 94%, 95%, 96%, 97%, 98%, or 99% sequence identity) to, or the sequence of, SEQ ID NO: 19. In further instance, the antibody reagent comprises a light chain variable domain (VL) sequence having at least 80% sequence identity (e.g., at least 81%, 82%, 83%, 84%, 85%, 86%, 87%, 88%, 89%, 90%, 91%, 92%, 93%, 94%, 95%, 96%, 97%, 98%, or 99% sequence identity) to, or the sequence of, SEQ ID NO: 20. In some instances, the antibody reagent comprises a VH sequence having at least 80% sequence identity (e.g., at least 81%, 82%, 83%, 84%, 85%, 86%, 87%, 88%, 89%, 90%, 91%, 92%, 93%, 94%, 95%, 96%, 97%, 98%, or 99% sequence identity) to, or the sequence of, SEQ ID NO: 19 and a VL sequence having at least 80% sequence identity (e.g., at least 81%, 82%, 83%, 84%, 85%, 86%, 87%, 88%, 89%, 90%, 91%, 92%, 93%, 94%, 95%, 96%, 97%, 98%, or 99% sequence identity) to, or the sequence of, SEQ ID NO: 20.

In some embodiments, the antibody reagent comprises a scFv comprising, in an N—C direction, a VH and a VL. In some embodiments, the scFv has an amino acid sequence at least 80% sequence identity (e.g., at least 81%, 82%, 83%, 84%, 85%, 86%, 87%, 88%, 89%, 90%, 91%, 92%, 93%, 94%, 95%, 96%, 97%, 98%, or 99% sequence identity) to, or the sequence of, SEQ ID NO: 21.

In some embodiments, the antibody reagent comprises a scFv comprising, in an N—C direction, a VL and a VH. In some embodiments, the scFv has an amino acid sequence at least 80% sequence identity (e.g., at least 81%, 82%, 83%, 84%, 85%, 86%, 87%, 88%, 89%, 90%, 91%, 92%, 93%, 94%, 95%, 96%, 97%, 98%, or 99% sequence identity) to, or the sequence of, SEQ ID NO: 22.

In some examples, any of the mesothelin antibody of the disclosure have one or more CDR (e.g., CDR-H or CDR-L) sequences substantially similar to any of the CDR-H1, CDR-H2, CDR-H3, CDR-L1, CDR-L2, and/or CDR-L3 sequences of the mesothelin antibody described herein. In some embodiments, the position of one or more CDRs along the VH (e.g., CDR-H1, CDR-H2, or CDR-H3) and/or VL (e.g., CDR-L1, CDR-L2, or CDR-L3) region of an antibody described herein can vary by one, two, three, four, five, or six amino acid positions so long as immunospecific binding to mesothelin is maintained (e.g., substantially maintained, for example, at least 50%, at least 60%, at least 70%, at least 80%, at least 90%, at least 95% of the binding of the original antibody). For example, in some embodiments, the position defining a CDR of the antibody described herein can vary by shifting the N-terminal and/or C-terminal boundary of the CDR by one, two, three, four, five, or six amino acids, relative to the CDR position so long as binding to mesothelin is maintained (e.g., substantially maintained, for example, at least 50%, at least 60%, at least 70%, at least 80%, at least 90%, at least 95% of the binding of the original antibody). In another embodiment, the length of one or more CDRs along the VH (e.g., CDR-H1, CDR-H2, or CDR-H3) and/or VL (e.g., CDR-L1, CDR-L2, or CDR-L3) region of an antibody described herein can vary (e.g., be shorter or longer) by one, two, three, four, five, or more amino acids, so long as binding to mesothelin is maintained (e.g., substantially maintained, for example, at least 50%, at least 60%, at least 70%, at least 80%, at least 90%, at least 95% of the binding of the original antibody).

In some embodiments, an antibody reagent comprises a CDR-L1, CDR-L2, CDR-L3, CDR-H1, CDR-H2, and/or CDR-H3 one, two, three, four, five or more amino acids shorter than one or more of the CDRs described herein (e.g., CDRs from an exemplary anti-mesothelin antibody described herein) so long as immunospecific binding to mesothelin is maintained (e.g., substantially maintained, for example, at least 50%, at least 60%, at least 70%, at least 80%, at least 90%, at least 95% relative to the binding of the original antibody). In some embodiments, an antibody reagent comprises a CDR-L1, CDR-L2, CDR-L3, CDR-HI, CDR-H2, and/or CDR-H3 that is one, two, three, four, five or more amino acids longer than one or more of the CDRs described herein (e.g., CDRs from an exemplary anti-mesothelin antibody described herein) so long as binding to mesothelin is maintained (e.g., substantially maintained, for example, at least 50%, at least 60%, at least 70%, at least 80%, at least 90%, at least 95% relative to the binding of the original antibody). In some embodiments, the amino portion of a CDR-L1, CDR-L2, CDR-L3, CDR-H1, CDR-H2, and/or CDR-H3 described herein can be extended by one, two, three, four, five or more amino acids compared to one or more of the CDRs described herein (e.g., CDRs from an exemplary anti-mesothelin antibody described herein) so long as binding to mesothelin is maintained (e.g., substantially maintained, for example, at least 50%, at least 60%, at least 70%, at least 80%, at least 90%, at least 95% relative to the binding of the original antibody). In some embodiments, the carboxy portion of a CDR-L1, CDR-L2, CDR-L3, CDR-H1, CDR-H2, and/or CDR-H3 described herein can be extended by one, two, three, four, five or more amino acids compared to one or more of the CDRs described herein (e.g., CDRs from an exemplary anti-mesothelin antibody described herein) so long as binding to mesothelin is maintained (e.g., substantially maintained, for example, at least 50%, at least 60%, at least 70%, at least 80%, at least 90%, at least 95% relative to the binding of the original antibody). In some embodiments, the amino portion of a CDR-L1, CDR-L2, CDR-L3, CDR-H1, CDR-H2, and/or CDR-H3 described herein can be shortened by one, two, three, four, five or more amino acids compared to one or more of the CDRs described herein (e.g., CDRs from an exemplary anti-mesothelin antibody described herein) so long as binding to mesothelin is maintained (e.g., substantially maintained, for example, at least 50%, at least 60%, at least 70%, at least 80%, at least 90%, at least 95% relative to the binding of the original antibody). In some embodiments, the carboxy portion of a CDR-L1, CDR-L2, CDR-L3, CDR-H1, CDR-H2, and/or CDR-H3 described herein can be shortened by one, two, three, four, five or more amino acids compared to one or more of the CDRs described herein (e.g., CDRs from an exemplary anti-mesothelin antibody described herein) so long as binding to mesothelin is maintained (e.g., substantially maintained, for example, at least 50%, at least 60%, at least 70%, at least 80%, at least 90%, at least 95% relative to the binding of the original antibody). Any method can be used to ascertain whether binding (e.g., immunospecific binding) to mesothelin is maintained, for example, using binding assays and conditions described in the art.

In a further aspect, an anti-mesothelin antibody reagent according to any of the above embodiments may incorporate any of the features, singly or in combination, as described in the sections below.

### Antibody Affinity

In some embodiments, an antibody reagent provided herein may have a dissociation constant (K_{D}) of ≤10 μM, ≤1 μM, ≤100 nM, ≤10 nM, ≤1 nM, ≤0.1 nM, or ≤0.01 nM. In one embodiment, the antibody reagent binds to mesothelin with a K_{D} between about 5 nM and about 1 μM. The affinity and binding kinetics of an antibody reagent (e.g., comprising an anti-mesothelin antibody or fragment thereof) can be tested using any suitable method including but not limited to biosensor technology (e.g., OCTET or BIACORE).

In one embodiment, K_{D} is measured by a radiolabeled antigen binding assay (RIA). In one embodiment, an RIA is performed with the Fab version of an antibody of interest and its antigen. For example, solution binding affinity of Fabs for antigen may be measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881 (1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™, Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{D} is measured using a BIACORE® surface plasmon resonance assay. For example, an assay using a BIACORE R-3000 (BIAcore, Inc., Piscataway, NJ) is performed at 25° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio kₒₙ/k_{off}. See, for example, Chen et al., J. Mol. Biol. 293:865-881 (1999). If the on-rate exceeds 10⁶ M-¹s-¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophotometer (Aviv Instruments) or a 8000-series SLM-AMINCO™ spectrophotometer (ThermoSpectronic) with a stirred cuvette.

### Antibody Fragments

In some embodiments, an antibody reagent provided herein comprises an antibody fragment. In some embodiments, the antibody fragment specifically binds to mesothelin or a portion thereof. In some embodiments, an antibody reagent comprises a first antibody fragment that specifically binds to mesothelin or a portion thereof, and one or more additional antibody fragments that specifically bind to a non-mesothelin antigen, e.g., a tumor-antigen or a T cell antigen. Antibody fragments include, but are not limited to, Fab, Fab′, Fab′-SH, F(ab′)₂, Fv, and scFv fragments, which are known in the art. Also included are diabodies, which have two antigen-binding sites that may be bivalent or bispecific, as is known in the art. Triabodies and tetrabodies are also known. Single-domain antibodies are also antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody as well as production by recombinant host cells (e.g., E. coli or phage), as described herein.

### Chimeric and Humanized Antibodies

In some embodiments, an antibody reagent provided herein is a chimeric antibody. The term “chimeric antibody” refers to either: (A) antibodies which comprise heavy and light chain variable region sequences from one species and constant region sequences from another species, such as antibodies having murine heavy and light chain variable regions linked to human constant regions; or (B) a “class switched” antibody in which the class or subclass has been changed from that of the parent antibody. In one example, a chimeric antibody comprises a non-human variable region (e.g., a variable region derived from a mouse, rat, hamster, rabbit, or non-human primate, such as a monkey) and a human constant region. Chimeric antibodies include antigen-binding fragments thereof.

In some embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

In some embodiments, an antibody reagent provided herein comprises a light chain variable domain that further comprises a light chain constant region. In some embodiments, the light chain constant region is a kappa, or a lambda light chain constant region. In some embodiments, the kappa or lambda light chain constant region is from a mammal, e.g., from a human, monkey, rat, or mouse. In some embodiments, the light chain constant region is a human kappa light chain constant region. In some embodiments, the light chain constant region is a human lambda light chain constant region. In some embodiments, the light chain constant region is a variant of any of the light chain constant regions provided herein. In some embodiments, the light chain constant region comprises an amino acid sequence that is at least 75%, 80%, 85%, 90%, 95%, 98%, or 99% identical to any of the light chain constant regions of an exemplary anti-mesothelin antibody reagent provided herein.

### Human Antibodies

In some embodiments, an antibody reagent provided herein is a human antibody (e.g., a human monoclonal antibody (HuMab), e.g., an anti-mesothelin HuMab). Human antibodies can be produced using various techniques known in the art.

Human monoclonal antibodies can be produced using a variety of known techniques, such as the standard somatic cell hybridization technique described by Kohler and Milstein, Nature 256:495 (1975). Although somatic cell hybridization procedures are preferred, in principle, other techniques for producing monoclonal antibodies also can be employed, e.g., viral or oncogenic transformation of B lymphocytes, phage display technique using libraries of human antibody genes.

In some instances, human antibodies are obtained by cloning the heavy and light chain genes directly from human B cells obtained from a human subject. The B cells are separated from peripheral blood (e.g., by flow cytometry, e.g., FACS), stained for B cell marker(s), and assessed for antigen binding. The RNA encoding the heavy and light chain variable regions (or the entire heavy and light chains) is extracted and reverse transcribed into DNA, from which the antibody genes are amplified (e.g., by PCR) and sequenced. The known antibody sequences can then be used to express recombinant human antibodies against a known target antigen (e.g., mesothelin).

In some instances, human antibodies may be prepared by administering an immunogen (e.g., mesothelin) to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. Human variable regions from intact antibodies generated by such animals may be further modified, for example, by combining with a different human constant region.

In some instances, human antibodies can also be made by hybridoma-based methods. The preferred animal system for generating hybridomas which produce human monoclonal antibodies is the murine system. Hybridoma production in the mouse is well known in the art, including immunization protocols and techniques for isolating and fusing immunized splenocytes. Human myeloma and mouse-human heteromyeloma cell lines for the production of human monoclonal antibodies have been described.

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Anti-mesothelin antibodies may be also produced using recombinant methods and compositions, for example, as described in U.S. Pat. No. 4,816,567. In one embodiment, isolated nucleic acid encoding an anti-mesothelin antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-mesothelin antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-mesothelin antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern.

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells, and myeloma cell lines such as Y0, NS0, and Sp2/0.

### Antibody Variants

In some embodiments, an antibody reagent may be an amino acid sequence variant of an anti-mesothelin antibody reagent described herein or known in the art. For example, it may be desirable to improve the binding affinity and/or other biological properties of an antibody reagent. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

In certain embodiments, antibody variants having one or more amino acid substitutions are provided. Sites of interest for substitutional mutagenesis include the CDRs and FRs. Conservative substitutions are shown in Table 2 under the heading of “preferred substitutions.” More substantial changes are provided in Table 2 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 2
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g., binding affinity).

Alterations (e.g., substitutions) may be made in CDRs, for example, to improve antibody affinity. Such alterations may be made in CDR “hotspots,” i.e., residues encoded by codons that undergo mutation at high frequency during the somatic maturation process, and/or residues that contact antigen, with the resulting variant VH or VL being tested for binding affinity. Affinity maturation by constructing and reselecting from secondary libraries is known in the art. In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two, or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, And Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

In certain embodiments, alternations may be made to the Fc region of an antibody. These alterations can be made alone, or in addition to, alterations to one or more of the antibody variable domains or regions thereof (e.g., one or more CDRs or FRs). The alterations to the Fc region may result in enhanced antibody effector functions (e.g., complement-dependent cytotoxicity (CDC)), for example, by increasing C1q avidity to opsonized cells. Exemplary mutations that enhance CDC include, for example, Fc mutations E345R, E430G, and S440Y. Accordingly, anti-mesothelin antibody reagents may contain one or more CDC-enhancing Fc mutations, which promote IgG hexamer formation and the subsequent recruitment and activation of C1, the first component of complement.

In certain embodiments, alterations of the amino acid sequences of the Fc region of the antibody may alter the half-life of the antibody in the host. Certain mutations that alter binding to the neonatal Fc receptor (FcRn) may extend half-life of antibodies in serum. For example, antibodies that have tyrosine in heavy chain position 252, threonine in position 254, and glutamic acid in position 256 of the heavy chain can have dramatically extended half-life in serum (see, e.g., U.S. Pat. No. 7,083,784).

In some embodiments, one, two or more mutations (e.g., amino acid substitutions) are introduced into the Fc region of an antibody described herein to alter one or more functional properties of the antibody, such as serum half-life, complement fixation, Fc receptor binding and/or antigen-dependent cellular cytotoxicity.

In some embodiments, one, two or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain can be altered to, e.g., facilitate assembly of the light and heavy chains, or to alter (e.g., increase or decrease) the stability of the antibody or to facilitate linker conjugation.

In some embodiments, one, two or more mutations (e.g., amino acid substitutions) are introduced into the Fc region of the antibody described herein to increase or decrease the affinity of the antibody for an Fc receptor (e.g., an activated Fc receptor) on the surface of an effector cell. Mutations in the Fc region of an antibody that decrease or increase the affinity of an antibody for an Fc receptor and techniques for introducing such mutations into the Fc receptor or fragment thereof are known to one of skill in the art. Examples of mutations in the Fc receptor of an antibody that can be made to alter the affinity of the antibody for an Fc receptor are described in, e.g., Smith P et al., (2012) PNAS 109:6181-6186, U.S. Pat. No. 6,737,056, and International Publication Nos. WO 02/060919; WO 98/23289; and WO 97/34631, which are incorporated herein by reference.

In some embodiments, one, two or more amino acid mutations (e.g., substitutions, insertions or deletions) are introduced into an IgG constant domain, or FcRn-binding fragment thereof (preferably an Fc or hinge-Fc domain fragment) to alter (e.g., decrease or increase) half-life of the antibody in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody in vivo.

In some embodiments, one, two or more amino acid substitutions are introduced into the Fc region to alter the effector function(s) of the antibody reagent (e.g., anti-mesothelin antibody reagent). This approach is described in further detail in U.S. Pat. Nos. 5,624,821 and 5,648,260. In some embodiments, the deletion or inactivation (through point mutations or other means) of a constant region domain can reduce Fc receptor binding of the circulating antibody thereby increasing tumor localization. See, e.g., U.S. Pat. Nos. 5,585,097 and 8,591,886 for a description of mutations that delete or inactivate the constant domain and thereby increase tumor localization. In some embodiments, one or more amino acid substitutions may be introduced into the Fc region of an antibody reagent described herein to remove potential glycosylation sites on Fc region, which may reduce Fc receptor binding (see, e.g., Shields R L et al., (2001) J Biol Chem 276:6591-604).

In some embodiments, one or more amino acids in the constant region of an antibody reagent described herein can be replaced with a different amino acid residue such that the antibody reagent has altered C1q binding and/or reduced or abolished complement dependent cytotoxicity (CDC). This approach is described in further detail in U.S. Pat. No. 6,194,551 (Idusogie et al). In some embodiments, one or more amino acid residues in the N-terminal region of the CH2 domain of an antibody reagent described herein are altered to thereby alter the ability of the antibody reagent to fix complement. This approach is described further in International Publication No. WO 94/29351. In some embodiments, the Fc region of an antibody reagent described herein is modified to increase the ability of the antibody reagent to mediate antibody dependent cellular cytotoxicity (ADCC) and/or to increase the affinity of the antibody for an Fcγ receptor. This approach is described further in International Publication No. WO 00/42072.

Alterations of amino acid sequence can be accomplished by any of a number of techniques known to one of skill in the art. Mutations can be introduced, for example, at particular loci by synthesizing oligonucleotides containing a mutant sequence, flanked by restriction sites permitting ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. U.S. Pat. Nos. 4,518,584 and 4,737,462, which are herein incorporated by reference in their entireties. Any cysteine residue not involved in maintaining the proper conformation of a polypeptide also can be substituted, generally with serine, to improve the oxidative stability of the molecule and prevent aberrant crosslinking. Conversely, cysteine bond(s) can be added to a polypeptide to improve its stability or facilitate oligomerization.

In some embodiments, an antibody reagent is modified, e.g., modified via glycosylation, phosphorylation, sumoylation, and/or methylation. In some embodiments, an antibody reagent is a glycosylated antibody, which is conjugated to one or more sugar or carbohydrate molecules. In some embodiments, the one or more sugar or carbohydrate molecule are conjugated to the antibody reagent via N-glycosylation, O-glycosylation, C-glycosylation, glypiation (GPI anchor attachment), and/or phosphoglycosylation. In some embodiments, the one or more sugar or carbohydrate molecule are monosaccharides, disaccharides, oligosaccharides, or glycans. In some embodiments, the one or more sugar or carbohydrate molecule is a branched oligosaccharide or a branched glycan. In some embodiments, the one or more sugar or carbohydrate molecule includes a mannose unit, a glucose unit, an N-acetylglucosamine unit, or a phospholipid unit. In some embodiments, there are about 1-10, about 1-5, about 5-10, about 1-4, about 1-3, or about 2 sugar molecules. In some embodiments, a glycosylated antibody is fully or partially glycosylated. In some embodiments, an antibody is glycosylated by chemical reactions or by enzymatic means. In some embodiments, an antibody is glycosylated in vitro or inside a cell, which may optionally be deficient in an enzyme in the N- or O-glycosylation pathway, e.g. a glycosyltransferase.

### Characterization of Monoclonal Antibodies to Mesothelin

Sequence information for human monoclonal antibodies of the disclosure can be ascertained using sequencing techniques which are well known in the art.

Similarly, affinity of the antibodies for mesothelin can also be assessed using standard techniques. For example, Biacore 3000 can be used to determine the affinity of HuMabs to mesothelin. HuMabs are captured on the surface of a Biacore chip (GE healthcare), for example, via amine coupling (Sensor Chip CM5). The captured HuMabs can be exposed to various concentrations of mesothelin in solution, and the Kₒₙ and K_{off} for an affinity (K_{D}) can be calculated, for example, by BIAevaluation software.

Human monoclonal antibodies of the disclosure can also be characterized for binding to mesothelin using a variety of known techniques, such as ELISA, Western blot, etc. Generally, the antibodies are initially characterized by ELISA. In some embodiments, an ELISA assay can be used to screen for antibodies and, thus, hybridomas that produce antibodies that show positive reactivity with the mesothelin immunogen. Hybridomas that bind, preferably with high affinity, to mesothelin can then be subcloned and further characterized. One clone from each hybridoma, which retains the reactivity of the parent cell (by ELISA), can then be chosen for making a cell bank, and for antibody purification.

In some embodiments, competition assays may be used to identify an antibody that competes with an anti-mesothelin antibody reagent of the disclosure for binding to mesothelin. In certain embodiments, such a competing antibody binds to the same epitope (e.g., a linear or a conformational epitope) that is bound by an anti-mesothelin antibody reagent of the disclosure. Detailed exemplary methods for mapping an epitope to which an antibody binds are provided in Morris (1996) “Epitope Mapping Protocols,” in Methods in Molecular Biology vol. 66 (Humana Press, Totowa, NJ).

In some aspects, the MGHmeso1 antibody reagent binds to an mesothelin epitope in Region III of mesothelin (Amino Acids 487-598). In some aspects, the MGHmeso1 antibody reagent binds to an mesothelin epitope comprising RLAFQNMNGSEY (SEQ ID NO: 37). In some embodiments, the MGH1meso1 antibody reagent binds to a different epitope than the SS1 antibody.

### Antibody Conjugates

In some embodiments, an anti-mesothelin antibody reagent of the disclosure can be covalently attached by linker (e.g., by a disulfide or non-cleavable thioether linker) to a moiety as an antibody conjugate. In some embodiments, the moiety is a drug moiety and the antibody conjugate is an antibody drug conjugate (ADC). In some embodiments, the moiety is a detectable moiety, e.g., facilitating the detection or quantification of the antibody conjugate. The drug to which the antibody is covalently attached may have cytotoxic or cytostatic effect when it is not conjugated to the antibody. The antibody-drug conjugate can be used to selectively delivery an effective dose of a cytotoxic agent to cells expressing mesothelin (e.g., to tumor tissue expressing mesothelin) in order to achieve greater selectivity and therefore a lower efficacious dose. By achieving higher selectivity, use of the antibody-drug conjugate may also reduce systemic exposure and increase tolerability of the drug. Additionally, the antibody-drug conjugate may improve the bioavailability of the drug and/or the antibody compared to when the drug and/or antibody is administered in its unconjugated form.

A variety of linker types and strategies are known in the art and any or all of these are contemplated for use with the antibody reagents of the disclosure. In some embodiments, the linker is biodegradable, e.g., cleavable by an endogenous protease (e.g., present in the target tissue and/or cells). In some embodiments the linker comprises a protease cleavable site. In some embodiments, the linker comprises a pH sensitive site, e.g., a site sensitive to acidic pH, e.g., that hydrolyzes under acidic conditions. In some embodiments, the linker is stable under physiological conditions, e.g., sufficiently stable so that the antibody targets the moiety to the target tissue (e.g., mesothelin-expressing tumor cells) prior to release of the moiety. In some embodiments, the linker comprises a disulfide bond, e.g., a glutathione-sensitive disulfide bond. In some embodiments, the drug moiety conjugated to the antibody reagent is active only after cleavage of the linker. In some embodiments, the drug moiety conjugated to the antibody reagent is active only after proteolytic digestion of the antibody reagent (e.g., in the lysosome of a target cell). In some embodiments, the linker is a non-cleavable heterobifunctional thiooether linker, e.g., a maleimide linker, e.g., comprising N-hydroxysuccinimide ester (succinimidyl-4-(N-maleimidomethyl)cyclohexane-1-carboxylate or SMCC.

A variety of drug moieties are known in the art and any or all of these are contemplated for use with the antibody reagents of the disclosure. In some embodiments, the drug comprises ozogamicin, vedotin, bleomycin, an auristatin, a maytansinoid, calicheamicin, emtansine, deruxtecan, govitecan, mafodotin, duocarmazine, soravtansine, tesirine, or a related chemical variant of any thereof (Joubert et al. Pharmaceuticals (Basel). 2020 September; 13 (9): 245).

A variety of detectable moieties are known in the art and any or all of these are contemplated for use with the antibody reagents of the disclosure. In some embodiments, a detectable moiety is chosen from a fluorescent molecule, a chromogenic molecule, an affinity purification tag, a radioisotope, a heavy isotope, a nucleotide probe, or a nanoparticle. In some embodiments, the detectable moiety is a fluorescent molecule, e.g., green fluorescent protein (GFP). In some embodiments, the detectable moiety is radiolabeled, e.g., comprises a radioisotope. In some embodiments, the detectable moiety is detectable by colorimetric techniques.

### Multispecific Antibody Reagents

In some embodiments, an antibody reagent is multispecific, capable of specifically binding two or more target antigens. In some embodiments, an antibody reagent is bispecific (i.e., a bispecific antibody reagent). In some embodiments, a bispecific antibody reagent specifically binds mesothelin and a second antigen. In some embodiments, the second antigen is a tumor antigen. In some embodiments, the second antigen is expressed by (e.g., on the surface of) mesothelin-expressing cancer cells. In some embodiments, the second antigen is epidermal growth factor receptor (EGFR), BCMA, CD19, CD37, carcinoembryonic antigen (CEA), epithelial cell adhesion molecule (EpCAM), EphA3, Her2/neu, MUC1, TGF-βRII, colony stimulating factor 1 receptor (CSF1R), prostate-specific membrane antigen (PSMA), or prostate stem cell antigen (PSCA).

A multispecific antibody reagent may comprise a first binding domain comprising a full-length antibody or antibody fragment thereof that binds to a first antigen (e.g., mesothelin) and a second binding domain comprising a different full-length antibody or antibody fragment thereof that binds to a second antigen (e.g., a tumor antigen). The first and second binding domains may be connected by a linker sequence. In some embodiments, the linker sequence is a flexible linker. For instance, linker sequences useful for the antibody reagents of the disclosure include, but are not limited to, glycine/serine linkers, e.g., GGGSGGGSGGGS (SEQ ID NO: 31) and Gly4Ser (G4S) (SEQ ID NO: 38) linkers such as (G4S) 3 (GGGGSGGGGSGGGGS (SEQ ID NO: 32)) and (G4S) 4 (GGGGSGGGGSGGGGSGGGGS (SEQ ID NO: 33)); the linker sequence of GSTSGSGKPGSGEGSTKG (SEQ ID NO: 34) as described by Whitlow et al., Protein Eng. 6(8): 989-95, 1993, the contents of which are incorporated herein by reference in its entirety; the linker sequence of GGSSRSSSSGGGGSGGGG (SEQ ID NO: 35) as described by Andris-Widhopf et al., Cold Spring Harb. Protoc. 2011(9), 2011, the contents of which are incorporated herein by reference in its entirety; as well as linker sequences with added functionalities, e.g., an epitope tag or an encoding sequence containing Cre-Lox recombination site as described by Sblattero et al., Nat. Biotechnol. 18(1): 75-80, 2000, the contents of which are incorporated herein by reference in its entirety.

### T Cell Engaging Antibody Molecules (TEAMs)

Also provided herein are bispecific antibody reagents known as T cell engaging antibody molecules (TEAMs) (also referred to in the literature as bispecific T cell engagers or BiTEs™). Such molecules can target T cells by binding to a T cell antigen (e.g., by binding CD3) as well as a target antigen, e.g., mesothelin. TEAMs can be used to augment T cell response in, e.g., the tumor microenvironment. In some embodiments, the T cell antigen is a Treg antigens. In some embodiments, the Treg antigen is selected from GARP, LAP, CD25, and CTLA-4. The two components of a TEAM can optionally be separated from one another by a linker as described herein (e.g., a glycine/serine-based linker), and may also be connected in either orientation, e.g., with the anti-CD3 component N-terminal to the anti-mesothelin component, or vice versa. In some embodiments, the antibody reagent targeting GARP comprises SEQ ID NO: 11. In some embodiments, the antibody reagent targeting GARP is a camelid antibody or single domain antibody. In some embodiments, the antibody reagent targeting LAP comprises SEQ ID NO: 12. In some embodiments, the antibody reagent targeting LAP comprises the VH and VL domain of SEQ ID NO: 12. In some embodiments, the antibody reagent targeting LAP comprises from N-terminal to C-terminal a VH domain then a VL domain. In some embodiments, the antibody reagent targeting LAP comprises from N-terminal to C-terminal a VL domain then a VH domain.

(SEQ ID NO: 11)
DIQMTQSPSSLSASLGDRVTITCQASQSISSYLAWYQQKPGQAPNILIY
GASRLKTGVPSRFSGSGSGTSFTLTISGLEAEDAGTYYCQQYASVPVTF
GQGTKVELK
(SEQ ID NO: 12)
MKLWLNWIFLVTLLNDIQCEVKLVESGGGLVQPGGSLSLSCAASGFTFT
DYYMSWVRQPPGKALEWLGFIRNKPNGYTTEYSASVKGRFTISRDNSQS
ILYLQMNVLRAEDSATYYCARYTGGGYFDYWGQGTTLTVSSGGGGSGGG
GSGGGGSGGGGSMMSSAQFLGLLLLCFQGTRCDIQMTQTTSSLSASLGD
RLTISCRASQDISNYLNWYQQKPDGTVKLLIYYTSRLHSGVPSRFSGSG
SGTDYSLTISNLEQADIATYFCQQGDTLPWTFGGGTKLEIK

An exemplary TEAM useful for the methods described herein includes a CD3-binding domain and a mesothelin-binding domain. Such a TEAM may include as the CD3-binding domain an anti-CD3 scFv. The anti-CD3 scFv may be derived from any anti-CD3 antibodies known in the art. The mesothelin-binding domain may comprise any antibody reagent that specifically binds mesothelin described herein, e.g., full-length antibodies or antibody fragments thereof (e.g., an antibody fragment that binds mesothelin).

For example, the TEAM may include a mesothelin-binding domain derived from an anti-mesothelin antibody as described above. In some embodiments, the mesothelin-binding domain includes a VH having at least 80% sequence identity (e.g., at least 81%, 82%, 83%, 84%, 85%, 86%, 87%, 88%, 89%, 90,% 91%, 92%, 93%, 94%, 95%, 96%, 97%, 98%, or 99% sequence identity) to, or the sequence of, SEQ ID NO: 19. In further embodiments, the mesothelin-binding domain includes a VL sequence having at least 80% sequence identity (e.g., at least 81%, 82%, 83%, 84%, 85%, 86%, 87%, 88%, 89%, 90,% 91%, 92%, 93%, 94%, 95%, 96%, 97%, 98%, or 99% sequence identity) to, or the sequence of, SEQ ID NO: 20. In certain embodiments, the mesothelin-binding domain includes a VH sequence having at least 80% sequence identity (e.g., at least 81%, 82%, 83%, 84%, 85%, 86%, 87%, 88%, 89%, 90,% 91%, 92%, 93%, 94%, 95%, 96%, 97%, 98%, or 99% sequence identity) to, or the sequence of, SEQ ID NO: 19 and a VL sequence having at least 80% sequence identity (e.g., at least 81%, 82%, 83%, 84%, 85%, 86%, 87%, 88%, 89%, 90,% 91%, 92%, 93%, 94%, 95%, 96%, 97%, 98%, or 99% sequence identity) to, or the sequence of, SEQ ID NO: 20.

The mesothelin-binding domain may be positioned N-terminal to the CD3-binding domain, or the CD3-binding domain may be positioned N-terminal to the mesothelin-binding domain. The mesothelin-binding domain and CD3-binding domain may optionally be connected via a linker sequence, e.g., a linker sequence of SEQ ID NO: 31-35 (described herein), as well as any other linker described herein or known in the art.

### Methods of Producing Antibodies

Appropriate methodologies may be used to obtain and/or produce antibodies, antibody fragments, or antigen-binding agents, e.g., through the use of recombinant DNA protocols. In some embodiments, an antibody may also be produced through the generation of hybridomas (see, e.g., Kohler, G and Milstein, C. “Continuous cultures of fused cells secreting antibody of predefined specificity” Nature, 1975, 256:495-497). The antigen-of-interest may be used as the immunogen in any form or entity, e.g., recombinant or a naturally occurring form or entity. Hybridomas are screened using standard methods, e.g., ELISA screening, to find at least one hybridoma that produces an antibody that targets a particular antigen. Antibodies may also be produced through screening of protein expression libraries that express antibodies, e.g., phage display libraries. Phage display library design may also be used, in some embodiments, (see, e.g. U.S. Pat. No. 5,223,409, filed Mar. 1, 1991, “Directed evolution of novel binding proteins”; WO 1992/18619, filed Apr. 10, 1992, “Heterodimeric receptor libraries using phagemids”; WO 1991/17271, filed May 1, 1991, “Recombinant library screening methods”; WO 1992/20791, filed May 15, 1992, “Methods for producing members of specific binding pairs”; WO 1992/15679, filed Feb. 28, 1992, and “Improved epitope displaying phage”). In some embodiments, an antigen-of-interest may be used to immunize a non-human animal, e.g., a rodent or a goat. In some embodiments, an antibody is then obtained from the non-human animal, and may be optionally modified using a number of methodologies, e.g., using recombinant DNA techniques. Additional examples of antibody production and methodologies are known in the art (see, e.g. Harlow et al. “Antibodies: A Laboratory Manual”, Cold Spring Harbor Laboratory, 1988.).

### Nucleic Acids Encoding Antibody Reagents

Also provided are nucleic acid constructs and vectors encoding the antibody reagents described herein. In some embodiments, nucleic acid constructs and vectors are used to express an antibody reagent in a cell. Efficient expression of antibody reagents in cells as described herein can be assessed using standard assays that detect the mRNA, DNA, or gene product of the nucleic acid encoding the proteins. For example, RT-PCR, FACS, northern blotting, western blotting, ELISA, or immunohistochemistry can be used. The antibody reagents described herein can be constitutively expressed or inducibly expressed. In some examples, the antibody reagents are encoded by a recombinant nucleic acid sequence.

In some embodiments, the polynucleotide sequence encoding the antibody reagent is operably linked to a promoter. The promoter can be a constitutively expressed promoter (e.g., an EF1α promoter) or an inducibly expressed promoter (e.g., a NFAT promoter).

Furthermore, the polynucleotides of the disclosure can include the expression of a suicide gene. This can be done to facilitate external, drug-mediated control of administered cells. For example, by use of a suicide gene, cells expressing the antibody reagent can be depleted, e.g., from the patient, in case of, e.g., an adverse event. In one example, the FK506 binding domain is fused to the caspase9 pro-apoptotic molecule. Cells (e.g., T cells) engineered in this manner are rendered sensitive to the immunosuppressive drug tacrolimus. Other examples of suicide genes are thymidine kinase (TK), CD20, thymidylate kinase, truncated prostate-specific membrane antigen (PSMA), truncated low affinity nerve growth factor receptor (LNGFR), truncated CD19, and modified Fas, which can be triggered for conditional ablation by the administration of specific molecules (e.g., ganciclovir to TK+ cells) or antibodies or antibody-drug conjugates.

Constructs including sequences encoding antibody reagents for expression in cells of the disclosure can be comprised within vectors. In some embodiments, a nucleic acid encoding a polypeptide as described herein (e.g., an antibody reagent) is comprised by a vector. In some of the aspects described herein, a nucleic acid sequence encoding a given polypeptide as described herein, or any module thereof, is operably linked to a vector. A selected nucleic acid sequence can be inserted into a vector and packaged in retroviral particles using techniques known in the art. The recombinant virus can then be isolated and delivered to cells, e.g., in vitro or ex vivo. In some embodiments, the vector is a viral vector or a non-viral vector. In some embodiments, the viral vector is a retroviral vector (e.g., a lentiviral vector), an adenovirus vector, or an adeno-associated virus vector.

In some embodiments, the vector is a retroviral vector. Retroviruses, such as lentiviruses, provide a convenient platform for delivery of nucleic acid sequences encoding a gene or chimeric gene of interest. A selected nucleic acid sequence can be inserted into a vector and packaged in retroviral particles using techniques known in the art. The recombinant virus can then be isolated and delivered to cells, e.g., in vitro or ex vivo. Retroviral systems are well known in the art and are described in, for example, U.S. Pat. No. 5,219,740; Kurth and Bannert (2010) “Retroviruses: Molecular Biology, Genomics and Pathogenesis” Calster Academic Press (ISBN: 978-1-90455-55-4); and Hu et al., Pharmacological Reviews 52:493-512, 2000; which are each incorporated by reference herein in their entirety. Lentiviral system for efficient DNA delivery can be purchased from OriGene; Rockville, MD.

In some embodiments, the antibody reagent described herein is expressed in a mammalian cell via transfection or electroporation of an expression vector comprising a nucleic acid encoding the antibody reagent. Transfection or electroporation methods are known in the art.

### Cells and Uses

One aspect of the technology described herein relates to a mammalian cell comprising any of the antibody reagents described herein; or a nucleic acid encoding any of the antibody reagents described herein. In one embodiment, the mammalian cell comprises an antibody reagent or a nucleic acid encoding such an antibody reagent. The mammalian cell or tissue can be of human, primate, hamster, rabbit, rodent, cow, pig, sheep, horse, goat, dog or cat origin, but any other mammalian cell may be used. In a preferred embodiment of any aspect, the mammalian cell is human.

In some embodiments of any aspect, the mammalian cell is an immune cell. As used herein, “immune cell” refers to a cell that plays a role in the immune response. Immune cells are of hematopoietic origin, and include lymphocytes, such as B cells and T cells; natural killer cells; myeloid cells, such as monocytes, macrophages, eosinophils, mast cells, basophils, and granulocytes. In some embodiments, the immune cell is a T cell; a NK cell; a NKT cell; lymphocytes, such as B cells and T cells; and myeloid cells, such as monocytes, macrophages, eosinophils, mast cells, basophils, and granulocytes. In one embodiment, the immune cell is a T cell.

In other embodiments, the immune cell is obtained from an individual having or diagnosed as having cancer, a plasma cell disorder, or autoimmune disease.

In some embodiments, the cell is an immortalized cell, e.g., of a cell line suitable for expression of a polypeptide (e.g., the antibody reagent). In some embodiments, the cell is a HEK293 or Chinese Hamster Ovary (CHO) cell. Without wishing to be bound by theory, an immortalized cell capable of expressing an antibody reagent described herein may be useful to produce desired quantities of the antibody reagent.

In another aspect, the disclosure provides a method of treating cancer in a subject comprising administering to a subject an antibody reagent described herein, a nucleic acid (e.g., a vector) encoding an antibody reagent described herein, a cell comprising an antibody reagent or nucleic acid described herein, or a pharmaceutical composition comprising any of the aforementioned. “Cancer” as used herein can refer to a hyperproliferation of cells whose unique trait, loss of normal cellular control, results in unregulated growth, lack of differentiation, local tissue invasion, and metastasis. Exemplary cancers include, but are not limited to, mesothelioma, pancreatic cancer, ovarian cancer, lung cancer, endometrial cancer, biliary cancer, gastric cancer, breast cancer, pleural cancer, colorectal cancer, appendiceal cancer, esophageal cancer, thymic cancer, or cervical cancer. Solid tumors can be found in bones, muscles, or organs, and can be sarcomas or carcinomas. It is contemplated that any aspect of the technology described herein can be used to treat all types of cancers, including cancers not listed in the instant application. As used herein, the term “tumor” refers to an abnormal growth of cells or tissues, e.g., of malignant type or benign type.

The efficacy of antibody reagents in, e.g., the treatment of a condition described herein, or to induce a response as described herein (e.g., a reduction in cancer cells) can be determined by the skilled clinician. However, a treatment is considered “effective treatment,” as the term is used herein, if one or more of the signs or symptoms of a condition described herein is altered in a beneficial manner, other clinically accepted symptoms are improved, or even ameliorated, or a desired response is induced, e.g., by at least 10% following treatment according to the methods described herein. Efficacy can be assessed, for example, by measuring a marker, indicator, symptom, and/or the incidence of a condition treated according to the methods described herein or any other measurable parameter appropriate. Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

Efficacy can also be measured by a failure of an individual to worsen as assessed by hospitalization, or failure to need medical interventions (i.e., progression of the disease is halted). Methods of measuring these indicators are known to those of skill in the art and/or are described herein.

Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy of a given approach can be assessed in animal models of a condition described herein. When using an experimental animal model, efficacy of treatment is evidenced when a statistically significant change in a marker is observed.

In another aspect, the disclosure provides a method of detecting a mesothelin protein in a sample, the method comprising contacting the sample with an antibody reagent or antibody conjugate described herein. In some embodiments, the antibody conjugate comprises a detectable moiety described herein, e.g., to facilitate to detection. In some embodiments, a method of detecting comprises performing a Western blot, immunohistochemistry, immunocytochemistry, Enzyme Linked Immunosorbent Assay, fluorescent microscopy, flow cytometry, or Fluorescence activated cell sorting (FACS) colorimetry. In some embodiments, the sample is taken from a subject, e.g., a human subject. In some embodiments, the sample comprises cells, e.g., cells taken from a tumor or suspected tumor, e.g., a biopsy sample. In another aspect, the disclosure provides a method of detecting an antibody conjugate described herein in a subject, the method comprising administering the antibody conjugate, a nucleic acid encoding the antibody conjugate, a cell comprising the antibody conjugate or nucleic acid encoding the same, or a pharmaceutical composition comprising any of the aforementioned to the subject. In some embodiments, the method of detecting comprises using magnetic resonance imagining, ultrasound, computerized tomography scan, positron emission tomography, or single-photon emission computerized tomography to detect the antibody conjugate in the subject. In some embodiments, a method of detecting a mesothelin protein described herein comprises collecting a sample from a subject (e.g., from a tumor or suspected tumor, e.g., a biopsy sample).

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior technology or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples, which in no way should be construed as being further limiting.

### EXAMPLES

The following are examples of useful methods and compositions. It is understood that various other embodiments may be practiced, given the description provided herein.

### Example 1: Generation and Characterization of an Exemplary Anti-Mesothelin Antibody Reagent

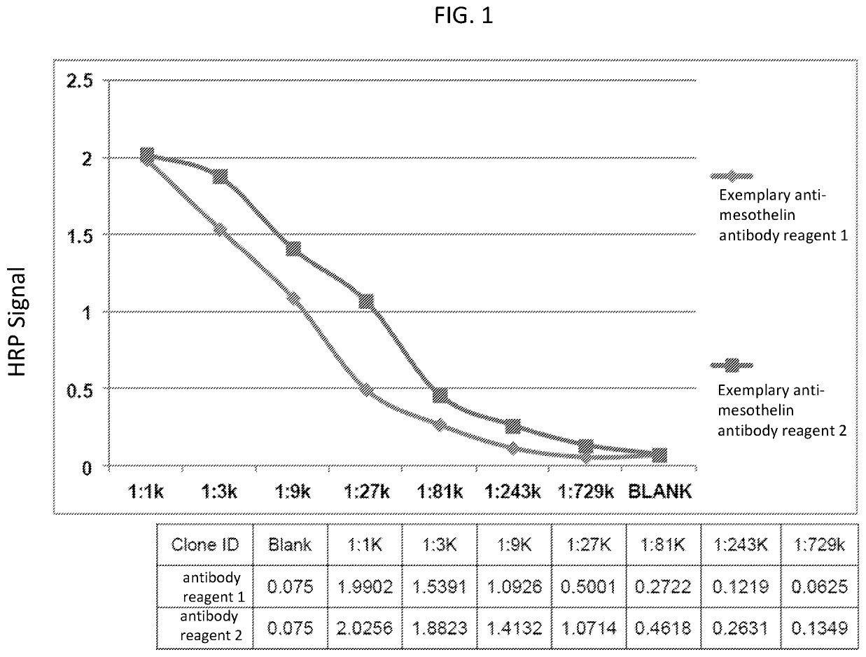

Mice were immunized with recombinant human mesothelin and two exemplary antibody reagents of the disclosure were produced. The two antibody reagents were assessed in an ELISA against mesothelin (2 μg/ml, 100 μl/well, in phosphate buffered saline pH 7.4) using a goat anti-mouse IgG conjugated to horse-radish peroxidase (HRP) (FIG. 1). The results showed that both antibodies bound to mesothelin. Exemplary antibody reagent 2 was selected for further study and designated MGHmeso1.

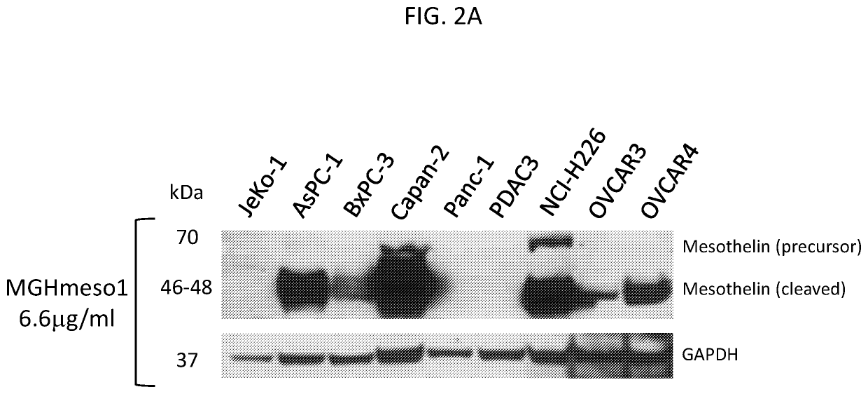

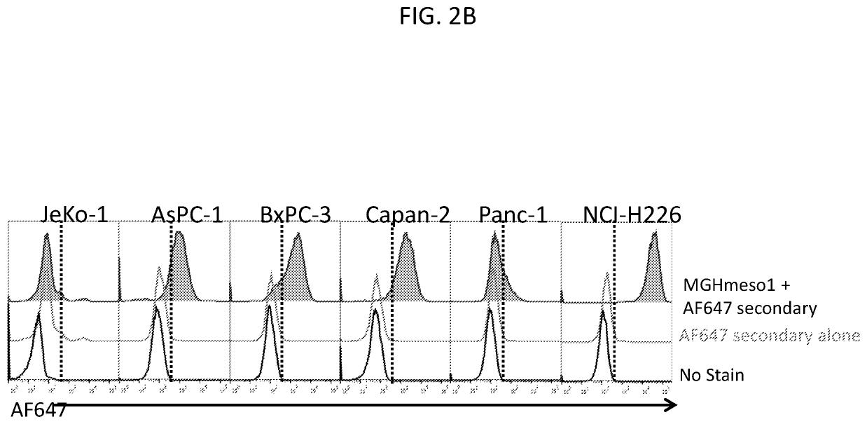

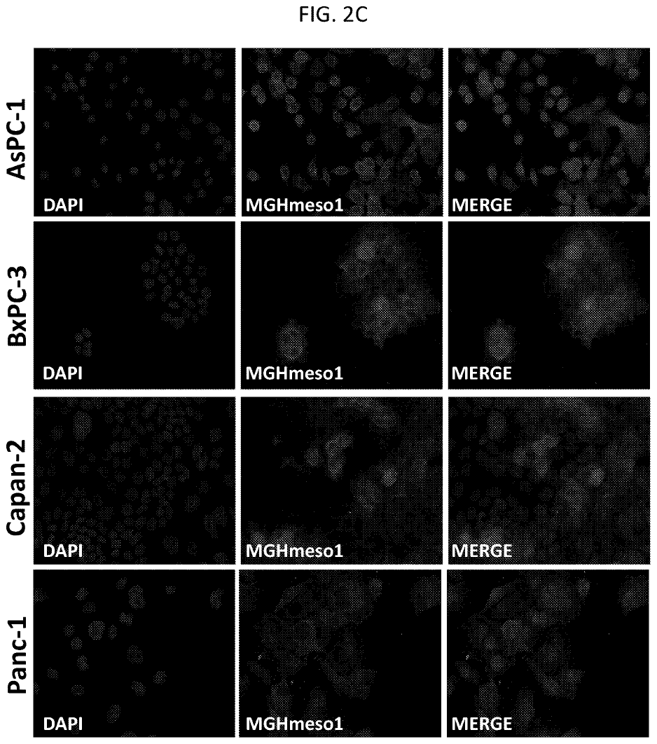

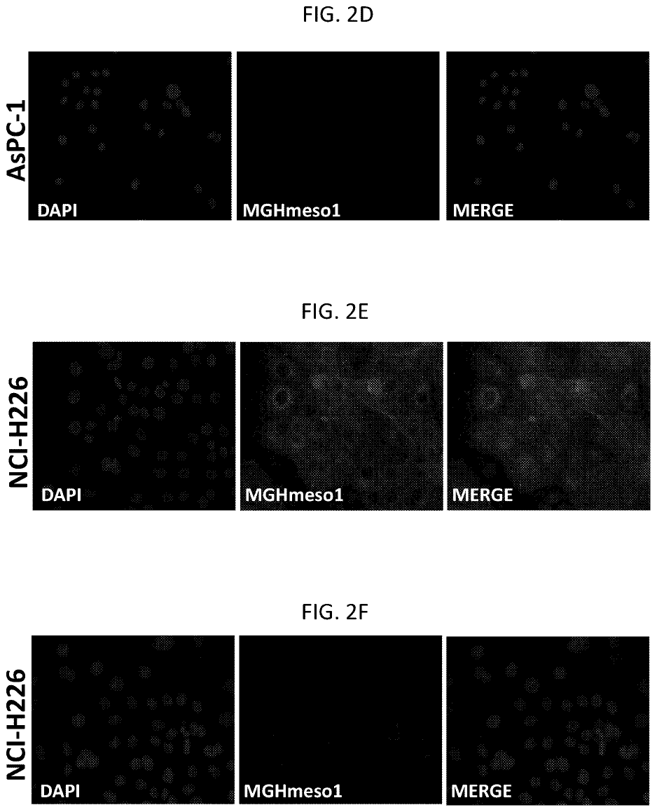

The capacity of MGHmeso1 to bind mesothelin expressing cells was evaluating by western blot (FIG. 2A) and flow cytometry (FIG. 2B). Lymphoma (JeKo-1), pancreatic (AsPC-1, BxPC-3, Capan-2, Panc-1, PDAC3), lung (NCI-H226) and ovarian (OVCAR3, OVCAR4) cell lines were expanded in culture, collected and used for western blot analysis. Protein was BCA quantified, loaded at 50 μg/lane and transferred to a PVDF membrane using semi-dry transfer. Membranes were probed with MGHmeso1 antibody (6.6 μg/ml) and GAPDH (1:10,000) overnight in TBST with 5% nonfat milk prior to washing and incubating with anti-mouse HRP secondary in 5% milk TBST for 1 hour at room temperature. JeKo-1 and PDAC3 were mesothelin negative control cells. The western blot results (FIG. 2A) demonstrated that MGHmeso1 recognizes mesothelin expressing cells and not mesothelin negative cells.

For flow cytometry analysis, cell lines were detached as needed using TrypLE and stained with MGHmeso1 antibody (0.66 μg/ml) for 20 minutes at room temperature. Cells were washed and incubated in anti-mouse AF647 IgG secondary antibody for 20 minutes at room temperature. After washing, cells were run on Fortessa and anaylzed using FlowJo software (FIG. 2B). JeKo-1 were mesothelin negative control cells. The results further confirmed that MGHmeso1 recognizes mesothelin expressing cells and not mesothelin negative cells.

Fluorescence microscopy was used to further characterize binding of MGHmeso1 to mesothelin-expressing cells: pancreatic cells (FIGS. 2C-2D) and lung cells (FIGS. 2E-2F). Pancreatic cell lines (AsPC-1, BxPC-3, Capan-2, Panc-1) and lung cell line (NCI-H226) were expanded in culture, detached using TrypLE and plated on iBidi glass-bottom microscopy slides for 2 days in culture. Cells were fixed, blocked and stained overnight using MGHmeso1 antibody (75 μg/ml) at 4° C. Slides were washed and incubated in anti-mouse AF647 secondary antibody at room temperature for 45 minutes before washing and sealing slides with Pro-long Gold Medium with DAPI. Slides were imaged using an Olympus microscope at 40×. As a control for specificity, AsPC-1 cells (FIG. 2D) or NCI-H226 cells (FIG. 2F) were given secondary antibody only. The results show that MGHmeso1 binds to pancreatic cell lines or lung cells which express mesothelin, further confirming that exemplary antibody reagent MGHmeso1 binds mesothelin and can recognize mesothelin-expressing cells.

### Example 2: CAR Constructs Comprising an Exemplary Anti-Mesothelin Antibody Reagent

An exemplary antibody reagent, MGHmeso1, comprising the amino acid sequences of SEQ ID NOs: 13-20 (Table 3) and generated and characterized in Example 1 was selected for testing as part of four CAR T constructs (SEQ ID NOs: 2-5).

TABLE 3
Sequences of CAR T Constructs Comprising Exemplary Antibody Reagent

| SEQ | | |
|---|---|---|
| ID NO: | Description | Sequence |
| 1 | anti- | MALPVTALLLPLALLLHAARPQVQLQQSGPELEKPGASVKISCK |
| | mesothelin | ASGYSFTGYTMNWVKQSHGKSLEWIGLITPYNGASSYNQKFRG |
| | SS1 CART | KATLTVDKSSSTAYMDLLSLTSEDSAVYFCARGGYDGRGFDY |
| | FAP TEAM | WGQGTTVTVSSGGGGSGGGGSGGGGSDIELTQSPAIMSASPGE |
| | | KVTMTCSASSSVSYMHWYQQKSGTSPKRWIYDTSKLASGVPGR |
| | | FSGSGSGNSYSLTISSVEAEDDATYYCQQWSGYPLTFGAGTKLE |
| | | ITTTPAPRPPTPAPTIASQPLSLRPEACRPAAGGAVHTRGLDFAC |
| | | DIYIWAPLAGTCGVLLLSLVITLYCKRGRKKLLYIFKQPFMRPV |
| | | QTTQEEDGCCRFPEEEEGGCELRVKFSRSADAPAYQQGQNQL |
| | | YNELNLGRREEYDVLDKRRGRDPEMGGKPRRKNPQEGLYNEL |
| | | QKDKMAEAYSEIGMKGERRRGKGHDGLYQGLSTATKDTYDAL |
| | | HMQALPPRSGGSGATNFSLLKQAGDVEENPGPMHMETDTLLL |
| | | WVLLLWVPGSTGDDIVMTQSPDSLAVSLGERATINCKSSQSLLY |
| | | SRNQKNYLAWYQQKPGQPPKLLIFWASTRESGVPDRFSGSGFG |
| | | TDFTLTISSLQAEDVAVYYCQQYFSYPLTFGQGTKVEIKGGGGS |
| | | GGGGSGGGGSQVQLVQSGAEVKKPGASVKVSCKTSRYTFTEYT |
| | | IHWVRQAPGQRLEWIGGINPNNGIPNYNQKFKGRVTITVDTSAS |
| | | TAYMELSSLRSEDTAVYYCARRRIAYGYDEGHAMDYWGQGTL |
| | | VTVSSGGGSDIKLQQSGAELARPGASVKMSCKTSGYTFTRYTM |
| | | HWVKQRPGQGLEWIGYINPSRGYTNYNQKFKDKATLTTDKSSS |
| | | TAYMQLSSLTSEDSAVYYCARYYDDHYCLDYWGQGTTLTVSS |
| | | VEGGSGGSGGSGGSGGVDDIQLTQSPAIMSASPGEKVTMTCRAS |
| | | SSVSYMNWYQQKSGTSPKRWIYDTSKVASGVPYRFSGSGSGTS |
| | | YSLTISSMEAEDAATYYCQQWSSNPLTFGAGTKLELKHHHHHH |
| | | SGGGGEGRGSLLTCGDVEENPGPRMVSKGEEDNMAIIKEFMRF |
| | | KVHMEGSVNGHEFEIEGEGEGRPYEGTQTAKLKVTKGGPLPFA |
| | | WDILSPQFMYGSKAYVKHPADIPDYLKLSFPEGFKWERVMNFE |
| | | DGGVVTVTQDSSLQDGEFIYKVKLRGTNFPSDGPVMQKKTMG |
| | | WEASSERMYPEDGALKGEIKQRLKLKDGGHYDAEVKTTYKAK |
| | | KPVQLPGAYNVNIKLDITSHNEDYTIVEQYERAEGRHSTGGMDE |
| | | YK |
| 2 | anti-Meso | MALPVTALLLPLALLLHAARPMECNWILPFILSVTSGVYSEILLQ |
| | H/L 4-1BB | QTGTVLARPGTSVKMSCKASGYTFTNYRMHWVKQRPGQGLE |
| | CAR | WIGGIYPGNSDTNYNQKFKDKAKLTAVTSTSTANMELSSLTNE |
| | | DSAVYYCLRGIRGSYFDYWGQGTTLTVSSGGGGSGGGGSGGG |
| | | GSGGGGSMETDTILLWVLLLWVPGSTGDIVLTQSPASLAVSLGQ |
| | | RATISCKASQSVDYDGDSYMNWYQQKPGQPPKLLIYAASNLES |
| | | GIPARFSGSGSGTDFTLNIHPVEEEDAATYYCQQSNEDPSTFGGG |
| | | TKLEVKTTTPAPRPPTPAPTIASQPLSLRPEACRPAAGGAVHTRG |
| | | LDFACDIYIWAPLAGTCGVLLLSLVITLYCKRGRKKLLYIFKQPF |
| | | MRPVQTTQEEDGCSCRFPEEEEGGCELRVKFSRSADAPAYQQG |
| | | QNQLYNELNLGRREEYDVLDKRRGRDPEMGGKPRRKNPQEGL |
| | | YNELQKDKMAEAYSEIGMKGERRRGKGHDGLYQGLSTATKDT |
| | | YDALHMQALPPRSGGGGEGRGSLLTCGDVEENPGPRMVSKGEE |
| | | DNMAIIKEFMRFKVHMEGSVNGHEFEIEGEGEGRPYEGTQTAK |
| | | LKVTKGGPLPFAWDILSPQFMYGSKAYVKHPADIPDYLKLSFPE |
| | | GFKWERVMNFEDGGVVTVTQDSSLQDGEFIYKVKLRGTNFPSD |
| | | GPVMQKKTMGWEASSERMYPEDGALKGEIKQRLKLKDGGHY |
| | | DAEVKTTYKAKKPVQLPGAYNVNIKLDITSHNEDYTIVEQYER |
| | | AEGRHSTGGMDELYK |
| 3 | anti-Meso | MALPVTALLLPLALLLHAARPMETDTILLWVLLLWVPGSTGDIV |
| | L/H 4-1BB | LTQSPASLAVSLGQRATISCKASQSVDYDGDSYMNWYQQKPGQ |
| | CAR | PPKLLIYAASNLESGIPARFSGSGSGTDFTLNIHPVEEEDAATYYC |
| | | QQSNEDPSTFGGGTKLEVKGGGGSGGGGSGGGGSGGGGSMEC |
| | | NWILPFILSVTSGVYSEILLQQTGTVLARPGTSVKMSCKASGYTF |
| | | TNYRMHWVKQRPGQGLEWIGGIYPGNSDTNYNQKFKDKAKLT |
| | | AVTSTSTANMELSSLTNEDSAVYYCLRGIRGSYFDYWGQGTTL |
| | | TVSSTTTPAPRPPTPAPTIASQPLSLRPEACRPAAGGAVHTRGLD |
| | | FACDIYIWAPLAGTCGVLLLSLVITLYCKRGRKKLLYIFKQPFMR |
| | | PVQTTQEEDGCSCRFPEEEEGGCELRVKFSRSADAPAYQQGQN |
| | | QLYNELNLGRREEYDVLDKRRGRDPEMGGKPRRKNPQEGLYN |
| | | ELQKDKMAEAYSEIGMKGERRRGKGHDGLYQGLSTATKDTYD |
| | | ALHMQALPPRSGGGGEGRGSLLTCGDVEENPGPRMVSKGEEDN |
| | | MAIIKEFMRFKVHMEGSVNGHEFEIEGEGEGRPYEGTQTAKLK |
| | | VTKGGPLPFAWDILSPQFMYGSKAYVKHPADIPDYLKLSFPEGF |
| | | KWERVMNFEDGGVVTVTQDSSLQDGEFIYKVKLRGTNFPSDGP |
| | | VMQKKTMGWEASSERMYPEDGALKGEIKQRLKLKDGGHYDA |
| | | EVKTTYKAKKPVQLPGAYNVNIKLDITSHNEDYTIVEQYERAEG |
| | | RHSTGGMDELYK |
| 4 | anti-Meso | MALPVTALLLPLALLLHAARPMECNWILPFILSVTSGVYSEILLQ |
| | H/L CD28 | QTGTVLARPGTSVKMSCKASGYTFTNYRMHWVKQRPGQGLE |
| | CAR | WIGGIYPGNSDTNYNQKFKDKAKLTAVTSTSTANMELSSLTNE |
| | | DSAVYYCLRGIRGSYFDYWGQGTTLTVSSGGGGSGGGGSGGG |
| | | GSGGGGSMETDTILLWVLLLWVPGSTGDIVLTQSPASLAVSLGQ |
| | | RATISCKASQSVDYDGDSYMNWYQQKPGQPPKLLIYAASNLES |
| | | GIPARFSGSGSGTDFTLNIHPVEEEDAATYYCQQSNEDPSTFGGG |
| | | TKLEVKTTTPAPRPPTPAPTIASQPLSLRPEACRPAAGGAVHTRG |
| | | LDFACDIYIWAPLAGTCGVLLLSLVITLYCRSKRSRLLHSDYMN |
| | | MTPRRPGPTRKHYQPYAPPRDFAAYRSRVKFSRSADAPAYQQG |
| | | QNQLYNELNLGRREEYDVLDKRRGRDPEMGGKPRRKNPQEGL |
| | | YNELQKDKMAEAYSEIGMKGERRRGKGHDGLYQGLSTATKDT |
| | | YDALHMQALPPRSGGGGEGRGSLLTCGDVEENPGPRMVSKGEE |
| | | DNMAIIKEFMRFKVHMEGSVNGHEFEIEGEGEGRPYEGTQTAK |
| | | LKVTKGGPLPFAWDILSPQFMYGSKAYVKHPADIPDYLKLSFPE |
| | | GFKWERVMNFEDGGVVTVTQDSSLQDGEFIYKVKLRGTNFPSD |
| | | GPVMQKKTMGWEASSERMYPEDGALKGEIKQRLKLKDGGHY |
| | | DAEVKTTYKAKKPVQLPGAYNVNIKLDITSHNEDYTIVEQYER |
| | | AEGRHSTGGMDELYK |
| 5 | anti-Meso | MALPVTALLLPLALLLHAARPMETDTILLWVLLLWVPGSTGDIV |
| | L/H CD28 | LTQSPASLAVSLGQRATISCKASQSVDYDGDSYMNWYQQKPGQ |
| | CAR | PPKLLIYAASNLESGIPARFSGSGSGTDFTLNIHPVEEEDAATYYC |
| | | QQSNEDPSTFGGGTKLEVKGGGGSGGGGSGGGGSGGGGSMEC |
| | | NWILPFILSVTSGVYSEILLQQTGTVLARPGTSVKMSCKASGYTF |
| | | TNYRMHWVKQRPGQGLEWIGGIYPGNSDTNYNQKFKDKAKLT |
| | | AVTSTSTANMELSSLTNEDSAVYYCLRGIRGSYFDYWGQGTTL |
| | | TVSSTTTPAPRPPTPAPTIASQPLSLRPEACRPAAGGAVHTRGLD |
| | | FACDIYIWAPLAGTCGVLLLSLVITLYCRSKRSRLLHSDYMNMT |
| | | PRRPGPTRKHYQPYAPPRDFAAYRSRVKFSRSADAPAYQQGQN |
| | | QLYNELNLGRREEYDVLDKRRGRDPEMGGKPRRKNPQEGLYN |
| | | ELQKDKMAEAYSEIGMKGERRRGKGHDGLYQGLSTATKDTYD |
| | | ALHMQALPPRSGGGGEGRGSLLTCGDVEENPGPRMVSKGEEDN |
| | | MAIIKEFMRFKVHMEGSVNGHEFEIEGEGEGRPYEGTQTAKLK |
| | | VTKGGPLPFAWDILSPQFMYGSKAYVKHPADIPDYLKLSFPEGF |
| | | KWERVMNFEDGGVVTVTQDSSLQDGEFIYKVKLRGTNFPSDGP |
| | | VMQKKTMGWEASSERMYPEDGALKGEIKQRLKLKDGGHYDA |
| | | EVKTTYKAKKPVQLPGAYNVNIKLDITSHNEDYTIVEQYERAEG |
| | | RHSTGGMDELYK |
| 6 | Sibrotuzumab | QVQLVQSGAEVKKPGASVKVSCKTSRYTFTEYTIHWVRQAPGQ |
| | VH | RLEWIGGINPNNGIPNYNQKFKGRVTITVDTSASTAYMELSSLRS |
| | | EDTAVYYCARRRIAYGYDEGHAMDYWGQGTLVTVSS |
| 7 | Sibrotuzumab | DIVMTQSPDSLAVSLGERATINCKSSQSLLYSRNQKNYLAWYQ |
| | VL | QKPGQPPKLLIFWASTRESGVPDRFSGSGFGTDFTLTISSLQAED |
| | | VAVYYCQQYFSYPLTFGQGTKVEIK |
| 8 | Sibrotuzumab | DIVMTQSPDSLAVSLGERATINCKSSQSLLYSRNQKNYLAWYQ |
| | sequence | QKPGQPPKLLIFWASTRESGVPDRFSGSGFGTDFTLTISSLQAED |
| | including | VAVYYCQQYFSYPLTFGQGTKVEIKGGGGSGGGGSGGGGSQV |
| | linker | QLVQSGAEVKKPGASVKVSCKTSRYTFTEYTIHWVRQAPGQRL |
| | | EWIGGINPNNGIPNYNQKFKGRVTITVDTSASTAYMELSSLRSED |
| | | TAVYYCARRRIAYGYDEGHAMDYWGQGTLVTVSS |
| 13 | MGHmeso1 | NYRMH |
| | antibody | |
| | CDR-H1 | |
| 14 | MGHmeso1 | GIYPGNSDTNYNQKFKD |
| | antibody | |
| | CDR-H2 | |
| 15 | MGHmeso1 | GIRGSYFDY |
| | antibody | |
| | CDR-H3 | |
| 16 | MGHmeso1 | KASQSVDYDGDSYMN |
| | antibody | |
| | CDR-L1 | |
| 17 | MGHmeso1 | AASNLES |
| | antibody | |
| | CDR-L2 | |
| 18 | MGHmeso1 | QQSNEDPST |
| | antibody | |
| | CDR-L3 | |
| 19 | MGHmeso1 | MECNWILPFILSVTSGVYSEILLQQTGTVLARPGTSVKMSCKAS |
| | antibody | GYTFTNYRMHWVKQRPGQGLEWIGGIYPGNSDTNYNQKFKDK |
| | VH | AKLTAVTSTSTANMELSSLTNEDSAVYYCLRGIRGSYFDYWGQ |
| | | GTTLTVSS |
| 20 | MGHmeso1 | METDTILLWVLLLWVPGSTGDIVLTQSPASLAVSLGQRATISCK |
| | antibody VL | ASQSVDYDGDSYMNWYQQKPGQPPKLLIYAASNLESGIPARFS |
| | | GSGSGTDFTLNIHPVEEEDAATYYCQQSNEDPSTFGGGTKLEVK |
| 21 | MGHmeso1 | MECNWILPFILSVTSGVYSEILLQQTGTVLARPGTSVKMSCKAS |
| | anti- | GYTFTNYRMHWVKQRPGQGLEWIGGIYPGNSDTNYNQKFKDK |
| | mesothelin | AKLTAVTSTSTANMELSSLTNEDSAVYYCLRGIRGSYFDYWGQ |
| | antibody | GTTLTVSSGGGGSGGGGSGGGGSGGGGSMETDTILLWVLLLW |
| | VH-VL | VPGSTGDIVLTQSPASLAVSLGQRATISCKASQSVDYDGDSYMN |
| | | WYQQKPGQPPKLLIYAASNLESGIPARFSGSGSGTDFTLNIHPVE |
| | | EEDAATYYCQQSNEDPSTFGGGTKLEVK |
| 22 | MGHmeso1 | METDTILLWVLLLWVPGSTGDIVLTQSPASLAVSLGQRATISCK |
| | anti- | ASQSVDYDGDSYMNWYQQKPGQPPKLLIYAASNLESGIPARFS |
| | mesothelin | GSGSGTDFTLNIHPVEEEDAATYYCQQSNEDPSTFGGGTKLEVK |
| | antibody | GGGGSGGGGSGGGGSGGGGSMECNWILPFILSVTSGVYSEILLQ |
| | VL-VH | QTGTVLARPGTSVKMSCKASGYTFTNYRMHWVKQRPGQGLE |
| | | WIGGIYPGNSDTNYNQKFKDKAKLTAVTSTSTANMELSSLTNE |
| | | DSAVYYCLRGIRGSYFDYWGQGTTLTVSS |
| 23 | mesothelin | MALPTARPLLGSCGTPALGSLLFLLFSLGWVQPSRTLAGETGQE |
| | protein | AAPLDGVLANPPNISSLSPRQLLGFPCAEVSGLSTERVRELAVAL |
| | sequence | AQKNVKLSTEQLRCLAHRLSEPPEDLDALPLDLLLFLNPDAFSG |
| | | PQACTRFFSRITKANVDLLPRGAPERQRLLPAALACWGVRGSLL |
| | | SEADVRALGGLACDLPGRFVAESAEVLLPRLVSCPGPLDQDQQ |
| | | EAARAALQGGGPPYGPPSTWSVSTMDALRGLLPVLGQPIIRSIPQ |
| | | GIVAAWRQRSSRDPSWRQPERTILRPRFRREVEKTACPSGKKAR |
| | | EIDESLIFYKKWELEACVDAALLATQMDRVNAIPFTYEQLDVLK |
| | | HKLDELYPQGYPESVIQHLGYLFLKMSPEDIRKWNVTSLETLKA |
| | | LLEVNKGHEMSPQAPRRPLPQVATLIDRFVKGRGQLDKDTLDT |
| | | LTAFYPGYLCSLSPEELSSVPPSSIWAVRPQDLDTCDPRQLDVLY |
| | | PKARLAFQNMNGSEYFVKIQSFLGGAPTEDLKALSQQNVSMDL |
| | | ATFMKLRTDAVLPLTVAEVQKLLGPHVEGLKAEERHRPVRDWI |
| | | LRQRQDDLDTLGLGLQGGIPNGYLVLDLSMQEALSGTPCLLGP |
| | | GPVLTVLALLLASTLA |
| 24 | CD8 | TTTPAPRPPTPAPTIASQPLSLRPEACRPAAGGAVHTRGLDFACD |
| | hinge/TM | IYIWAPLAGTCGVLLLSLVITLYC |
| | domain | |
| 25 | 4-1 BB ICD | KRGRKKLLYIFKQPFMRPVQTTQEEDGCSCRFPEEEEGGCEL |
| 26 | CD3-zeta | RVKFSRSADAPAYQQGQNQLYNELNLGRREEYDVLDKRRGRD |
| | ICD-1 | PEMGGKPRRKNPQEGLYNELQKDKMAEAYSEIGMKGERRRGK |
| | | GHDGLYQGLSTATKDTYDALHMQALPPR |
| 27 | CD3-zeta | RVKFSRSADAPAYQQGQNQLYNELNLGRREEYDVLDKRRGRD |
| | ICD-2 | PEMGGKPRRKNPQEGLYNELQKDKMAEAYSEIGMKGERRRGK |
| | | GHDGLYQGLSTATKDTYDALHMQALPPR |
| 28 | CD28 ICD | RSKRSRLLHSDYMNMTPRRPGPTRKHYQPYAPPRDFAAYRS |
| 29 | CD8 leader | MALPVTALLLPLALLLHAARP |
| 30 | IgK signal | METDTLLLWVLLLWVPGSTGD |
| | sequence | |
| 31 | linker-1 | GGGSGGGSGGGS |
| | (G3S)3 | |
| 32 | linker-2 | GGGGSGGGGSGGGGS |
| | (G4S)3 | |
| 33 | linker-3 | GGGGSGGGGSGGGGSGGGGS |
| | (G4S)4 | |
| 34 | linker-4 | GSTSGSGKPGSGEGSTKG |
| 35 | linker-5 | GGSSRSSSSGGGGSGGGG |
| 36 | T2A | SGGGGEGRGSLLTCGDVEENPGPR |

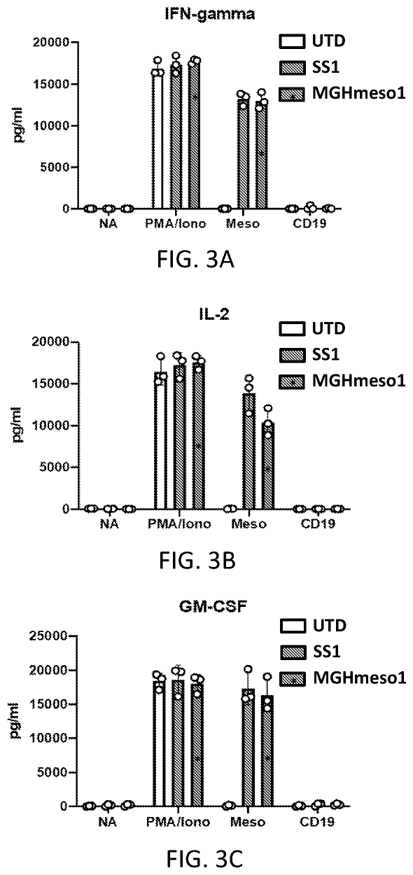

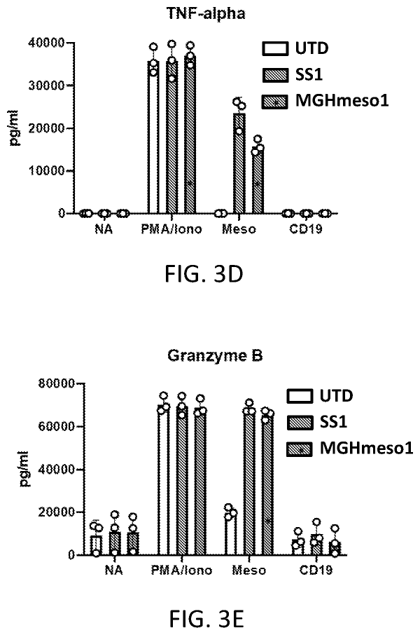

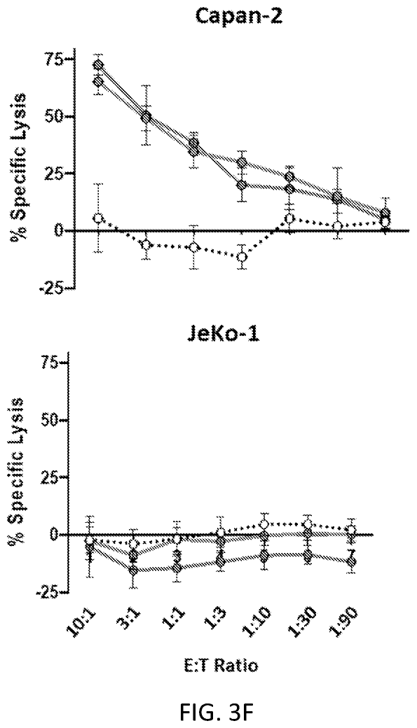

The cytokine expression profile of exemplary mesothelin CAR T cells comprising a CAR comprising the exemplary antibody reagent, MGHmeso1, was compared to the cytokine expression profile of a CAR T cell comprising a CAR comprising a previously known anti-mesothelin antibody, SS1 (FIGS. 3A-3E). T cells were collected from 3 normal donors, activated, transduced with plasmid encoding a CAR comprising SS1 or a plasmid encoding a CAR comprising an exemplary anti-mesothelin antibody reagent of the disclosure, MGHmeso1, and expanded in IL-2 for 10 days. FIG. 3A-3E compare cytokine production in these T cells, as well as untransfected cells. Results showed that expression of both the exemplary mesothelin CAR comprising MGHmeso1 as well as the CAR comprising SS1 greatly increased expression of INF-gamma, IL-2, GM-CSF, TNF-alpha, and Granzyme B in the CAR T cells. To confirm antigen-specific killing, cell lysis was assessed in an overnight (22 hour) luciferase-based killing assay with Mesothelin+ pancreatic cancer (Capan-2) or Mesothelin-lymphoma cells (JeKo-1) cell lines. N=3. Results showed that both CAR T cells specifically lysed mesothelin positive Capan-2 cells, but did not lyse mesothelin negative JeKo-1 cells (Mantle cell lymphoma) (FIGS. 3E-3F). The results from FIGS. 3A-3E demonstrate that exemplary mesothelin CAR T cells comprising a CAR comprising the exemplary antibody reagent MGHmeso1 exhibit similar cell function and cytotoxicity as those employing previously known anti-mesothelin antibodies, illustrating the utility of the anti-mesothelin antibody reagents of the disclosure.

### Example 3: Determining the MGHmeso1 Antibody Reagent Mesothelin Epitope

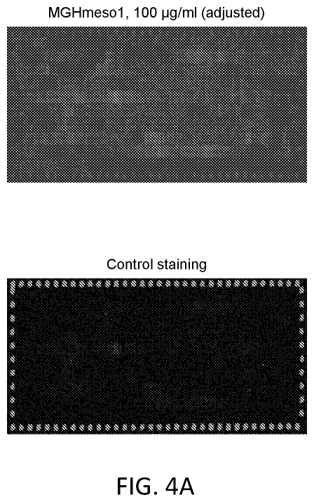

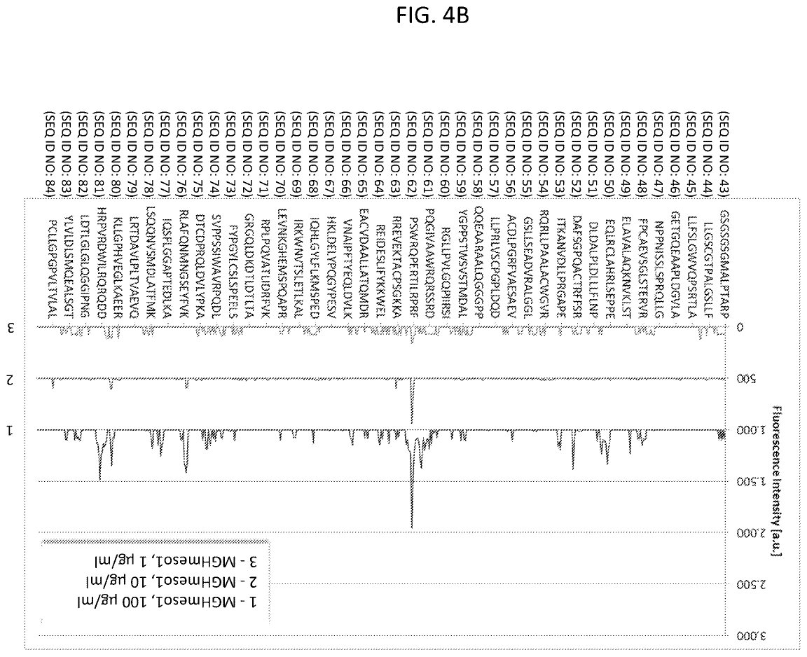

MGHmeso1 epitope binding sites were determined using PEPperMAP® Epitope mapping using micro-array binding (FIG. 4A). Briefly, 630 mesothelin peptide fragments were produced for determining the MGHmeso1 epitope. The peptide fragments were all 15 amino acids in length with a 14 amino acid peptide-peptide overlap. These were printed into peptide microarrays in duplicate. HA peptides were also printed into the microarrays and used as controls. A solution containing the MGHmeso1 antibody was washed over the microarray and MGHmeso1 binding was detected based on fluorescence using a secondary antibody that binds to MGHmeso1.

Results showed multiple possible MGHmeso1 epitopes: LSEPPED (SEQ ID NO: 39), GPQACTRFFSRITK (SEQ ID NO: 40), WRQPERTILRPRF (SEQ ID NO: 41), RLAFQNMNGSEY (SEQ ID NO: 37) and DWILRQ (SEQ ID NO: 42) (FIG. 4B). Based on the clarity associated with the microarray spot morphologies, RLAFQNMNGSEY (SEQ ID NO: 37) is expected to be the MGHMeso1 binding epitope. RLAFQNMNGSEY (SEQ ID NO: 37) is located in Region III (amino acids 487-598) of mesothelin. The different regions of mesothelin are described by Zhang et al. Scientific Reports 5.1 (2015): 1-14.

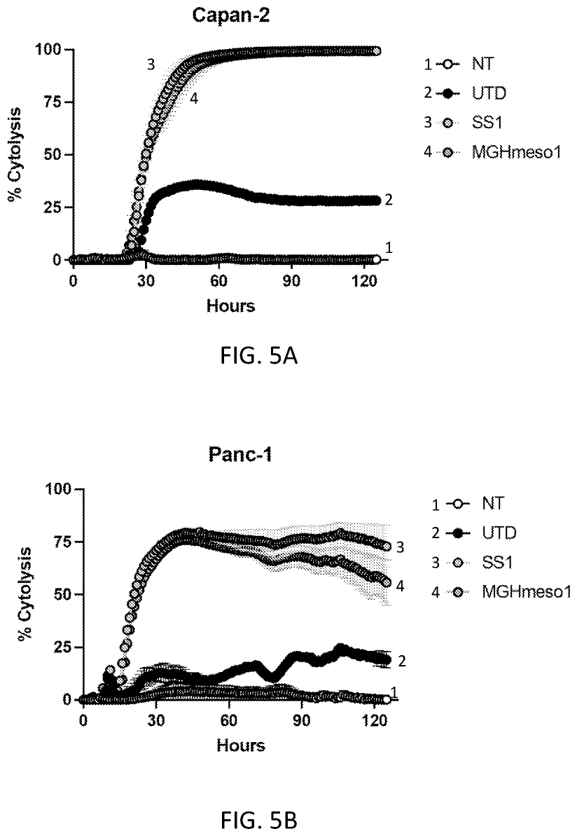

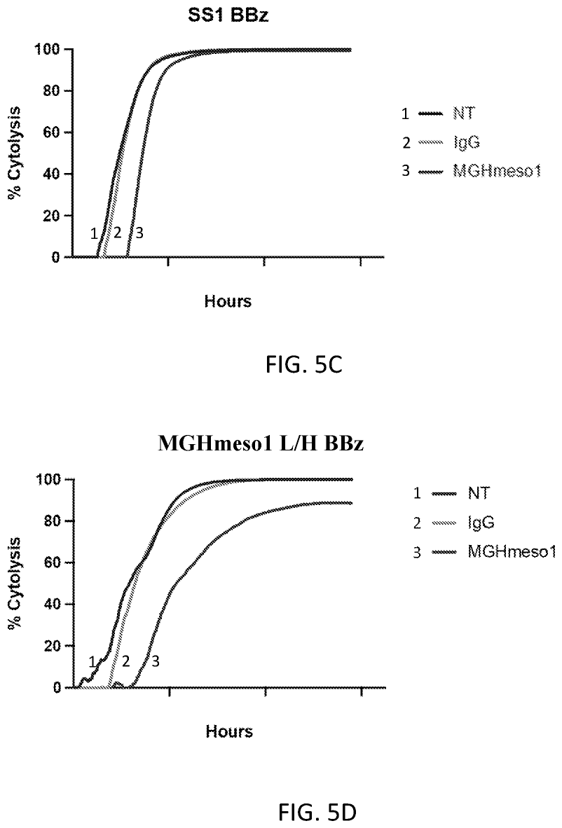

Further experiments provided evidence that the SS1 antibody and the MGHmeso1 antibody have similar effects when used in CARs and bind to different mesothelin epitopes. The effects of a SSI containing CAR T cells (SS1 BBz CAR T cells) and an MGHmeso1 containing CAR-T cells (MGHmeso1 L/H BBz CAR T cells) were tested against two different cancer cells lines, Capan-2 and Panc-1 (both pancreatic cancers). FIGS. 5A-5B. Results show that the SS1 BBz CAR T cells and an MGHmeso1 containing CAR-T cell have similar cytotoxicity. To determine if SS1 and MGHmeso1 bind to the same epitope, the SS1 BBz CAR T cells and the MGHmeso1 L/H BBz CAR T cells were administered in the presence of MGHmeso1 antibody. Results show that MGHmeso1 antibody decreases the cytotoxicity of the MGHmeso1 L/H BBz CAR T cells, but not the SS1 BBz CAR T cells, which indicates that MGHmeso1 and SS1 bind to different mesothelin epitopes.

What is claimed is:

1. An antibody reagent that binds to mesothelin or a portion thereof, comprising:
(a) a heavy chain variable domain (VH) comprising three complementarity determining regions CDR-H1, CDR-H2, and CDR-H3, wherein the CDR-H1 comprises an amino acid sequence of SEQ ID NO: 13, or an amino acid sequence with no more than 1, 2, or 3 amino acid substitutions of SEQ ID NO: 13; the CDR-H2 comprises an amino acid sequence of SEQ ID NO: 14, or an amino acid sequence with no more than 1, 2, or 3 amino acid substitutions of SEQ ID NO: 14; and the CDR-H3 comprises an amino acid sequence of SEQ ID NO: 15, or an amino acid sequence with no more than 1, 2, or 3 amino acid substitutions of SEQ ID NO: 15, and
(b) a light chain variable domain (VL) comprising three complementarity determining regions CDR-L1, CDR-L2, and CDR-L3, wherein the CDR-L1 comprises an amino acid sequence of SEQ ID NO: 16, or an amino acid sequence with no more than 1, 2, or 3 amino acid substitutions of SEQ ID NO: 16; the CDR-L2 comprises an amino acid sequence of SEQ ID NO: 17, or an amino acid sequence with no more than 1, 2, or 3 amino acid substitutions of SEQ ID NO: 17; and the CDR-L3 comprises an amino acid sequence of SEQ ID NO: 18, or an amino acid sequence with no more than 1, 2, or 3 amino acid substitutions of SEQ ID NO: 18.

2. The antibody reagent of claim 1, wherein the antibody reagent comprises an antibody or an antibody fragment.

3. The antibody reagent of claim 1 or claim 2, wherein the antibody reagent is selected from the group consisting of an immunoglobulin molecule, a recombinant antibody, a disulfide linked Fv, a camelid antibody, a monoclonal antibody, a polyclonal antibody, a single-chain variable fragment (scFv), a chimeric antibody, a single domain antibody, a CDR-grafted antibody, a bispecific antibody, a diabody, an antigen binding fragment (Fab), a F(ab′)2, and a Fv.

4. The antibody reagent of any one of claims 1-3, wherein the antibody is a human antibody or a humanized antibody.

5. The antibody reagent of any one of claims 1-4, wherein the antibody reagent is an scFv.

6. The antibody reagent of any one of claims 1-5, comprising:
(a) a heavy chain variable domain (VH) comprising three complementarity determining regions CDR-H1, CDR-H2, and CDR-H3, wherein the CDR-H1 comprises an amino acid sequence of SEQ ID NO: 13; the CDR-H2 comprises an amino acid sequence of SEQ ID NO: 14; and the CDR-H3 comprises an amino acid sequence of SEQ ID NO: 15; and
(b) a light chain variable domain (VL) comprising three complementarity determining regions CDR-L1, CDR-L2, and CDR-L3, wherein the CDR-L1 comprises an amino acid sequence of SEQ ID NO: 16; the CDR-L2 comprises an amino acid sequence of SEQ ID NO: 17; and the CDR-L3 comprises an amino acid sequence of SEQ ID NO: 18.

7. The antibody reagent of any one of claims 1-6, comprising:
(a) a heavy chain variable domain (VH) comprising three complementarity determining regions CDR-H1, CDR-H2, and CDR-H3, wherein the CDR-H1 consists of an amino acid sequence of SEQ ID NO: 13; the CDR-H2 consists of an amino acid sequence of SEQ ID NO: 14; and the CDR-H3 consists of an amino acid sequence of SEQ ID NO: 15; and
(b) a light chain variable domain (VL) comprising three complementarity determining regions CDR-L1, CDR-L2, and CDR-L3, wherein the CDR-L1 consists of an amino acid sequence of SEQ ID NO: 16; the CDR-L2 consists of an amino acid sequence of SEQ ID NO: 17; and the CDR-L3 consists of an amino acid sequence of SEQ ID NO: 18.

8. The antibody reagent of any one of claims 1-7, comprising a VH domain of the amino acid sequence of SEQ ID NO: 19 or an amino acid sequence having at least 90% sequence identity to the amino acid sequence of SEQ ID NO: 19.

9. The antibody reagent of any one of claims 1-8, comprising a VL domain of the amino acid sequence of SEQ ID NO: 20, or an amino acid sequence having at least 90% sequence identity to the amino acid sequence of SEQ ID NO: 20.

10. The antibody reagent of any one of claims 1-9, comprising a VH-VL domain of the amino acid sequence of SEQ ID NO: 21, or an amino acid sequence having at least 90% sequence identity to the amino acid sequence of SEQ ID NO: 21.

11. The antibody reagent of any one of claims 1-9, comprising a VL-VH domain of the amino acid sequence of SEQ ID NO: 22, or an amino acid sequence having at least 90% sequence identity to the amino acid sequence of SEQ ID NO: 22.

12. The antibody reagent of any one of claims 1-11, wherein the mesothelin protein comprises an amino acid sequence of SEQ ID NO: 23.

13. A bispecific antibody reagent comprising the antibody reagent of any one of claims 1-12 and a second antibody reagent.

14. The bispecific antibody reagent of claim 13, wherein the second antibody reagent binds to a tumor antigen.

15. The bispecific antibody reagent of claim 14, wherein the tumor antigen to which the second antibody reagent binds to a solid tumor antigen.

16. The bispecific antibody reagent of claim 13, wherein the second antibody reagent binds to a protein selected from the group consisting of: CD3, epidermal growth factor (EGFR), and a portion of either thereof.

17. The bispecific antibody reagent of any of claims 13-16, comprising a cleavable moiety between the antibody reagent of any one of claims 1-12 and the second antibody reagent.

18. An antibody conjugate comprising the antibody reagent of any one of claims 1-12 or the bi-specific antibody reagent of any one of claims 13-17 and a conjugate moiety.

19. The antibody conjugate of claim 18, wherein the conjugate moiety is a cytotoxic agent.

20. The antibody conjugate of claim 19, wherein the cytotoxic agent is selected from the group consisting of ozogamicin, vedotin, bleomycin, an auristatin, a maytansinoid, calicheamicin, emtansine, deruxtecan, govitecan, mafodotin, duocarmazine, soravtansine, and tesirine.

21. The antibody conjugate of claim 18, wherein the conjugate is a detectable moiety.

22. The antibody conjugate of claim 21, wherein the detectable moiety comprises a fluorescent molecule, a chromogenic molecule, an affinity purification tag, a radioisotope, a heavy isotope, a nucleotide probe, or a nanoparticle.

23. A nucleic acid molecule comprising a nucleic acid sequence encoding any one of the antibody reagents of claims 1-12 or any one of the bispecific antibodies of claim 13-17.

24. The nucleic acid molecule of claim 23, wherein the nucleic acid molecule is a vector or a plasmid.

25. A viral vector comprising the nucleic acid molecule of claim 23 or claim 24.

26. The viral vector of claim 25, wherein the viral vector is an adenovirus associated vector, a lentiviral vector, or an oncolytic herpes simplex virus.

27. A cell comprising the antibody reagent of any one of claims 1-12, the bispecific antibody reagent of any one of claims 13-17, the antibody conjugate of claims 18-22 or the nucleic acid molecule of any one of claims 23-24.

28. The cell of claim 27, wherein the cell is a human cell.

29. The cell of claim 28, wherein the human cell is an immune cell.

30. The cell of any one of claims 27-29, wherein the cell is a cancer cell.

31. A composition comprising the antibody reagent of any one of claims 1-12, the bispecific antibody reagent of any one of claims 13-17, the antibody conjugate of claims 18-22, the nucleic acid molecule of any one of claims 23-24, or the cell of any one of claims 27-30.

32. A composition comprising a therapeutically effective amount of the antibody reagent of any one of claims 1-12, the bispecific antibody reagent of any one of claims 13-17, the antibody conjugate of claims 18-22 or the nucleic acid molecule of any one of claims 23-24, the viral vector of any one of claims 25-26, or the cell of any one of claims 27-30.

33. The composition of claim 31 or claim 32, further comprising a pharmaceutically acceptable excipient.

34. A method of treating cancer in a subject comprising administering to the subject the antibody reagent of any one of claims 1-12, the bispecific antibody reagent of any one of claims 13-17, the antibody conjugate of claims 18-22, the nucleic acid molecule of any one of claims 23-24, the viral vector of any one of claims 25-26, the cell of any one of claims 27-30, or the composition of any one of claims 31-33.

35. The method of claim 34, wherein the cancer is characterized by the presence of one or more solid tumors.

36. The method of claim 34 or claim 35, wherein the cancer expresses mesothelin.

37. The method of any one of claims 34-36, wherein the cancer is selected from the group consisting of mesothelioma, pancreatic cancer, ovarian cancer, lung cancer, endometrial cancer, biliary cancer, breast cancer, pleural cancer, colorectal cancer, appendiceal cancer, esophageal cancer, thymic cancer, cervical cancer, and gastric cancer.

38. The method of any one of claims 34-37, wherein the subject is a human.

39. A method of detecting a mesothelin protein in a sample, comprising contacting the sample with the antibody conjugate of claim 21 or claim 22.

40. The method of claim 39, further comprising contacting the antibody conjugate with a sample.

41. The method of claim 40, wherein the method of detecting the mesothelin protein comprises performing a Western blot, immunohistochemistry, immunocytochemistry, Enzyme Linked Immunosorbent Assay, fluorescent microscopy, flow cytometry, or Fluorescence activated cell sorting (FACS) colorimetry.

42. The method of claim 40 or claim 41, wherein the sample comprises mesothelin.

43. The method of claim 40, further comprising administering the antibody conjugate to a subject.

44. The method of claim 43, comprising detecting the antibody conjugate in the subject using magnetic resonance imagining, ultrasound, computerized tomography scan, positron emission tomography, or single-photon emission computerized tomography.

45. The method of claim 43, further comprising collecting a sample from the subject.

46. The method of claim 45, further comprising contacting the antibody conjugate with the sample.

47. The method of claim 46, wherein the method of detecting the mesothelin protein comprises performing a Western blot, immunohistochemistry, immunocytochemistry, Enzyme Linked Immunosorbent Assay, fluorescent microscopy, flow cytometry, or Fluorescence activated cell sorting (FACS) colorimetry.

48. The antibody reagent of any one of claims 1-12, wherein the antibody reagent binds to an epitope in region III of mesothelin.

49. The antibody reagent of claim 48, wherein the antibody reagent binds to an epitope comprising the amino acid sequence of RLAFQNMNGSEY (SEQ ID NO: 37).


SEQUENCE LISTING (84 sequences)

SEQ ID NO: 1 - protein, 1294 aa, synthetic construct
MALPVTALLLPLALLLHAARPQVQLQQSGPELEKPGASVKISCKASGYSFTGYTMNWVKQSHGKSLEWIGLITPYNGASSYNQKFRGKATLTVDKSSSTAYMDLLSLTSEDSAVYFCARGGYDGRGFDYWGQGTTVTVSSGGGGSGGGGSGGGGSDIELTQSPAIMSASPGEKVTMTCSASSSVSYMHWYQQKSGTSPKRWIYDTSKLASGVPGRFSGSGSGNSYSLTISSVEAEDDATYYCQQWSGYPLTFGAGTKLEITTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGSGATNFSLLKQAGDVEENPGPMHMETDTLLLWVLLLWVPGSTGDDIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKGGGGSGGGGSGGGGSQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSSGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELKHHHHHHSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDEYK

SEQ ID NO: 2 - protein, 792 aa, synthetic construct
MALPVTALLLPLALLLHAARPMECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSSGGGGSGGGGSGGGGSGGGGSMETDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 3 - protein, 792 aa, synthetic construct
MALPVTALLLPLALLLHAARPMETDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVKGGGGSGGGGSGGGGSGGGGSMECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 4 - protein, 791 aa, synthetic construct
MALPVTALLLPLALLLHAARPMECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSSGGGGSGGGGSGGGGSGGGGSMETDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVKTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 5 - protein, 791 aa, synthetic construct
MALPVTALLLPLALLLHAARPMETDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVKGGGGSGGGGSGGGGSGGGGSMECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSSTTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSGGGGEGRGSLLTCGDVEENPGPRMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 6 - protein, 124 aa, synthetic construct
QVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 7 - protein, 113 aa, synthetic construct
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIK

SEQ ID NO: 8 - protein, 252 aa, synthetic construct
DIVMTQSPDSLAVSLGERATINCKSSQSLLYSRNQKNYLAWYQQKPGQPPKLLIFWASTRESGVPDRFSGSGFGTDFTLTISSLQAEDVAVYYCQQYFSYPLTFGQGTKVEIKGGGGSGGGGSGGGGSQVQLVQSGAEVKKPGASVKVSCKTSRYTFTEYTIHWVRQAPGQRLEWIGGINPNNGIPNYNQKFKGRVTITVDTSASTAYMELSSLRSEDTAVYYCARRRIAYGYDEGHAMDYWGQGTLVTVSS

SEQ ID NO: 9-10 - skipped (2 entries)

SEQ ID NO: 11 - protein, 107 aa, synthetic construct
DIQMTQSPSSLSASLGDRVTITCQASQSISSYLAWYQQKPGQAPNILIYGASRLKTGVPSRFSGSGSGTSFTLTISGLEAEDAGTYYCQQYASVPVTFGQGTKVELK

SEQ ID NO: 12 - protein, 286 aa, synthetic construct
MKLWLNWIFLVTLLNDIQCEVKLVESGGGLVQPGGSLSLSCAASGFTFTDYYMSWVRQPPGKALEWLGFIRNKPNGYTTEYSASVKGRFTISRDNSQSILYLQMNVLRAEDSATYYCARYTGGGYFDYWGQGTTLTVSSGGGGSGGGGSGGGGSGGGGSMMSSAQFLGLLLLCFQGTRCDIQMTQTTSSLSASLGDRLTISCRASQDISNYLNWYQQKPDGTVKLLIYYTSRLHSGVPSRFSGSGSGTDYSLTISNLEQADIATYFCQQGDTLPWTFGGGTKLEIK

SEQ ID NO: 13 - protein, 5 aa, synthetic construct
NYRMH

SEQ ID NO: 14 - protein, 17 aa, synthetic construct
GIYPGNSDTNYNQKFKD

SEQ ID NO: 15 - protein, 9 aa, synthetic construct
GIRGSYFDY

SEQ ID NO: 16 - protein, 15 aa, synthetic construct
KASQSVDYDGDSYMN

SEQ ID NO: 17 - protein, 7 aa, synthetic construct
AASNLES

SEQ ID NO: 18 - protein, 9 aa, synthetic construct
QQSNEDPST

SEQ ID NO: 19 - protein, 137 aa, synthetic construct
MECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSS

SEQ ID NO: 20 - protein, 131 aa, synthetic construct
METDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVK

SEQ ID NO: 21 - protein, 288 aa, synthetic construct
MECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSSGGGGSGGGGSGGGGSGGGGSMETDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVK

SEQ ID NO: 22 - protein, 288 aa, synthetic construct
METDTILLWVLLLWVPGSTGDIVLTQSPASLAVSLGQRATISCKASQSVDYDGDSYMNWYQQKPGQPPKLLIYAASNLESGIPARFSGSGSGTDFTLNIHPVEEEDAATYYCQQSNEDPSTFGGGTKLEVKGGGGSGGGGSGGGGSGGGGSMECNWILPFILSVTSGVYSEILLQQTGTVLARPGTSVKMSCKASGYTFTNYRMHWVKQRPGQGLEWIGGIYPGNSDTNYNQKFKDKAKLTAVTSTSTANMELSSLTNEDSAVYYCLRGIRGSYFDYWGQGTTLTVSS

SEQ ID NO: 23 - protein, 630 aa, Homo sapiens
MALPTARPLLGSCGTPALGSLLFLLFSLGWVQPSRTLAGETGQEAAPLDGVLANPPNISSLSPRQLLGFPCAEVSGLSTERVRELAVALAQKNVKLSTEQLRCLAHRLSEPPEDLDALPLDLLLFLNPDAFSGPQACTRFFSRITKANVDLLPRGAPERQRLLPAALACWGVRGSLLSEADVRALGGLACDLPGRFVAESAEVLLPRLVSCPGPLDQDQQEAARAALQGGGPPYGPPSTWSVSTMDALRGLLPVLGQPIIRSIPQGIVAAWRQRSSRDPSWRQPERTILRPRFRREVEKTACPSGKKAREIDESLIFYKKWELEACVDAALLATQMDRVNAIPFTYEQLDVLKHKLDELYPQGYPESVIQHLGYLFLKMSPEDIRKWNVTSLETLKALLEVNKGHEMSPQAPRRPLPQVATLIDRFVKGRGQLDKDTLDTLTAFYPGYLCSLSPEELSSVPPSSIWAVRPQDLDTCDPRQLDVLYPKARLAFQNMNGSEYFVKIQSFLGGAPTEDLKALSQQNVSMDLATFMKLRTDAVLPLTVAEVQKLLGPHVEGLKAEERHRPVRDWILRQRQDDLDTLGLGLQGGIPNGYLVLDLSMQEALSGTPCLLGPGPVLTVLALLLASTLA

SEQ ID NO: 24 - protein, 69 aa, synthetic construct
TTTPAPRPPTPAPTIASQPLSLRPEACRPAAGGAVHTRGLDFACDIYIWAPLAGTCGVLLLSLVITLYC

SEQ ID NO: 25 - protein, 42 aa, synthetic construct
KRGRKKLLYIFKQPFMRPVQTTQEEDGCSCRFPEEEEGGCEL

SEQ ID NO: 26 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 112 aa, synthetic construct; identical to SEQ ID NO: 26

SEQ ID NO: 28 - protein, 41 aa, synthetic construct
RSKRSRLLHSDYMNMTPRRPGPTRKHYQPYAPPRDFAAYRS

SEQ ID NO: 29 - protein, 21 aa, synthetic construct
MALPVTALLLPLALLLHAARP

SEQ ID NO: 30 - protein, 21 aa, synthetic construct
METDTLLLWVLLLWVPGSTGD

SEQ ID NO: 31 - protein, 12 aa, synthetic construct
GGGSGGGSGGGS

SEQ ID NO: 32 - protein, 15 aa, synthetic construct
GGGGSGGGGSGGGGS

SEQ ID NO: 33 - protein, 20 aa, synthetic construct
GGGGSGGGGSGGGGSGGGGS

SEQ ID NO: 34 - protein, 18 aa, synthetic construct
GSTSGSGKPGSGEGSTKG

SEQ ID NO: 35 - protein, 18 aa, synthetic construct
GGSSRSSSSGGGGSGGGG

SEQ ID NO: 36 - protein, 24 aa, synthetic construct
SGGGGEGRGSLLTCGDVEENPGPR

SEQ ID NO: 37 - protein, 12 aa, synthetic construct
RLAFQNMNGSEY

SEQ ID NO: 38 - protein, 5 aa, synthetic construct
GGGGS

SEQ ID NO: 39 - protein, 7 aa, synthetic construct
LSEPPED

SEQ ID NO: 40 - protein, 14 aa, synthetic construct
GPQACTRFFSRITK

SEQ ID NO: 41 - protein, 13 aa, synthetic construct
WRQPERTILRPRF

SEQ ID NO: 42 - protein, 6 aa, synthetic construct
DWILRQ

SEQ ID NO: 43 - protein, 15 aa, synthetic construct
GSGSGSGMALPTARP

SEQ ID NO: 44 - protein, 15 aa, synthetic construct
LLGSCGTPALGSLLF

SEQ ID NO: 45 - protein, 15 aa, synthetic construct
LLFSLGWVQPSRTLA

SEQ ID NO: 46 - protein, 15 aa, synthetic construct
GETGQEAAPLDGVLA

SEQ ID NO: 47 - protein, 15 aa, synthetic construct
NPPNISSLSPRQLLG

SEQ ID NO: 48 - protein, 15 aa, synthetic construct
FPCAEVSGLSTERVR

SEQ ID NO: 49 - protein, 15 aa, synthetic construct
ELAVALAQKNVKLST

SEQ ID NO: 50 - protein, 15 aa, synthetic construct
EQLRCLAHRLSEPPE

SEQ ID NO: 51 - protein, 15 aa, synthetic construct
DLDALPLDLLLFLNP

SEQ ID NO: 52 - protein, 15 aa, synthetic construct
DAFSGPQACTRFFSR

SEQ ID NO: 53 - protein, 15 aa, synthetic construct
ITKANVDLLPRGAPE

SEQ ID NO: 54 - protein, 15 aa, synthetic construct
RQRLLPAALACWGVR

SEQ ID NO: 55 - protein, 15 aa, synthetic construct
GSLLSEADVRALGGL

SEQ ID NO: 56 - protein, 15 aa, synthetic construct
ACDLPGRFVAESAEV

SEQ ID NO: 57 - protein, 15 aa, synthetic construct
LLPRLVSCPGPLDQD

SEQ ID NO: 58 - protein, 15 aa, synthetic construct
QQEAARAALQGGGPP

SEQ ID NO: 59 - protein, 15 aa, synthetic construct
VGPPSTWSVSTMDAL

SEQ ID NO: 60 - protein, 15 aa, synthetic construct
RGLLPVLGQPIIRSI

SEQ ID NO: 61 - protein, 15 aa, synthetic construct
PQGIVAAWRQRSSRD

SEQ ID NO: 62 - protein, 15 aa, synthetic construct
PSWRQPERTILRPRF

SEQ ID NO: 63 - protein, 15 aa, synthetic construct
RREVEKTACPSGKKA

SEQ ID NO: 64 - protein, 15 aa, synthetic construct
REIDESLIFYKKWEL

SEQ ID NO: 65 - protein, 15 aa, synthetic construct
EACVDAALLATQMDR

SEQ ID NO: 66 - protein, 15 aa, synthetic construct
VNAIPFTYEQLDVLK

SEQ ID NO: 67 - protein, 15 aa, synthetic construct
HKLDELYPQGYPESV

SEQ ID NO: 68 - protein, 15 aa, synthetic construct
IQHLGYLFLKMSPED

SEQ ID NO: 69 - protein, 15 aa, synthetic construct
IRKWNVTSLETLKAL

SEQ ID NO: 70 - protein, 15 aa, synthetic construct
LEVNKGHEMSPQAPR

SEQ ID NO: 71 - protein, 15 aa, synthetic construct
RPLPQVATLIDRFVK

SEQ ID NO: 72 - protein, 15 aa, synthetic construct
GRGQLDKDTLDTLTA

SEQ ID NO: 73 - protein, 15 aa, synthetic construct
FYPGYLCSLSPEELS

SEQ ID NO: 74 - protein, 15 aa, synthetic construct
SVPPSSIWAVRPQDL

SEQ ID NO: 75 - protein, 15 aa, synthetic construct
DTCDPRQLDVLYPKA

SEQ ID NO: 76 - protein, 15 aa, synthetic construct
RLAFQNMNGSEYFVK

SEQ ID NO: 77 - protein, 15 aa, synthetic construct
IQSFLGGAPTEDLKA

SEQ ID NO: 78 - protein, 15 aa, synthetic construct
LSQQNVSMDLATFMK

SEQ ID NO: 79 - protein, 15 aa, synthetic construct
LRTDAVLPLTVAEVQ

SEQ ID NO: 80 - protein, 15 aa, synthetic construct
KLLGPHVEGLKAEER

SEQ ID NO: 81 - protein, 15 aa, synthetic construct
HRPVRDWILRQRQDD

SEQ ID NO: 82 - protein, 15 aa, synthetic construct
LDTLGLGLQGGIPNG

SEQ ID NO: 83 - protein, 15 aa, synthetic construct
YLVLDLSMQEALSGT

SEQ ID NO: 84 - protein, 15 aa, synthetic construct
PCLLGPGPVLTVLAL